(19) United States
(12) Patent Application Publication
(10) Pub. No.: US 2005/0065732 A1
(43) Pub. Date: Mar. 24, 2005

(54) Matrix methods for quantitatively analyzing and assessing the properties of botanical samples

(76) Inventors: Robert Tilton, Guilford, CT (US); Jeff Bjoraker, New York, NY (US); Jingdong Xu, Boston, MA (US)

Correspondence Address: MORGAN LEWIS & BOCKIUS LLP, 1111 PENNSYLVANIA AVENUE NW, WASHINGTON, DC, 20004, US

(21) Appl. No.: 10/493,574

(22) PCT Filed: Oct. 25, 2002
(86) PCT No.: PCT/US02/34121

Related U.S. Application Data
(60) Provisional application No. 60/330,628, filed on Oct. 26, 2001.

(51) Int. Cl. G06F 19/00; G01N 33/48; G01N 33/50

(52) U.S. Cl. 702/19

(57) ABSTRACT

This invention relates to computational methodologies for improving the selection, testing, quality control, and manufacture of herbal compositions, and to help guide the development of new herbal compositions and identify novel uses of existing herbal compositions. More specifically, this invention relates to a process of encoding two or more biological and/or chemical data into a matrix fingerprint, and the statistical/probabilistic manipulation of such matrix fingerprints for the testing and improvement of herbal compositions.

### RELATED APPLICATIONS

This application claims priority to U.S. Provisional Patent Application Ser. No. 60/330,628, filed on Oct. 26, 2001, which is herein incorporated in it entirety. This application is related to U.S. Provisional Application Ser. Nos. 60/105,435 and 60/188,021, PCT Application Nos. PCT/US99/24851 and PCT/US01/07608, and U.S. application Ser. No. 09/830,033. These applications are herein incorporated by reference in their entirety.

### FIELD OF THE INVENTION

This invention relates to computational methodologies for improving the selection, testing, quality control, and manufacture of herbal compositions. More specifically, this invention relates to a process of encoding two or more biological and/or chemical data points into a matrix fingerprint coding for a pattern of inter-relationships between the data points, and the statistical/probabilistic manipulation of such matrix fingerprints for the assessment, testing and improvement of herbal compositions. This invention also allows for the computation of a histogram of values for each data point or a single average value or a range of deterministic values that can be used to quantitatively assess similarities and differences between botanical samples. This value or set of values may then be used to assess reproducibility, define component composition, assess component modifications and enhance component optimization of pharmaceutically active botanicals or herbal medicines. These methods can be applied to either a multicomponent mixture such as those inherent in botanicals or herbal medicines or for multifactorial responses resulting from the testing or treatment with a single compound or a multicomponent mixture.

### BACKGROUND OF THE INVENTION

All publications and patent applications cited herein are incorporated by reference to the same extent as if each individual publication or patent application was specifically and individually indicated to be incorporated by reference.

Herbal medicine has been in use for centuries by the indigenous peoples of the Americas, Asia, Africa and Europe. In the United States (US), herbs have become commercially valuable in the dietary supplement industry as well as in holistic medicine. Approximately one third of the US population has tried some form of alternative medicine at least once (Eisenberg et al., 1993, N. Engl. J. Med. 328:246-252).

Botanicals, including herbs, have also become a focal point for the identification of new active agents to treat diseases. Active compounds, derived from plant extracts, are of continuing interest to the pharmaceutical industry. For example, taxol is an antineoplastic drug obtained from the bark of the western yew tree. It is estimated that approximately 30-35 percent of the thousands of drugs commonly used and prescribed today are either derived from a plant source or contain chemical imitations of a plant compound.

Currently, a number of medicinal formulations, food supplements, dietary supplements and the like contain herbal components or extracts from herbs. Herbal medicines have been used for treating various diseases of humans and animals in many different countries for a very long period of time (see, e.g., I. A. Ross, 1999, Medicinal Plants of the World, Chemical Constituents, Traditional and Modern Medicinal Uses, Humana Press; D. Molony, 1998, The American Association of Oriental Medicine's Complete Guide to Chinese Herbal Medicine, Berkley Books; Kessler et al., 1996, The Doctor's Complete Guide to Healing Medicines, Berkley Health/Reference Books; Mindell, supra).

The study of botanical extracts, however, provides unique challenges to perform both qualitative and, more importantly, quantitative analyses and comparisons. Some of the challenges include variability in the multicomponent mixtures of phytochemicals inherent in agricultural techniques, differences in manufacturing protocols, aging and shelf-life of the botanical drug and little reliable information regarding the pharmacologically active set of molecules. There is currently inadequate or poor quantitative methods to monitor and measure chemical and/or biological equivalence of botanical medicine compositions.

United States Regulatory Process. Currently, botanicals are treated as food or neutraceutical products. In the US, dietary supplements (such as botanical extracts and products, vitamins and minerals, amino acids and tissue extracts) are regulated under the Dietary Supplement Health and Education Act of 1994 (the DSHE Act). This Act removed the ingredients of dietary supplements from regulation as food additives under the Federal Food, Drug, and Cosmetic Act. In addition, the DSHE Act requires that The Food and Drug Administration (FDA) bear the burden of proof that a marketed dietary supplement presents a serious or unreasonable risk under the conditions of use on the label or as commonly consumed. Thus, there are currently no federal regulations that establish specific criteria for purity, identification and manufacturing procedures for dietary supplements. In addition, few published papers on herbal quality have resulted from the establishment of the Office of Alternative Medicine by Congress in 1992 (Angell et al., 1998, N. Engl. J. Med. 339:839-841).

At the present time, the FDA must approve each one of the chemical entities in a drug composition or cocktail, and then clinical trials must be undertaken so as to obtain separate FDA approval for marketing the drug. This process is extremely tedious and costly. A molecular holistic medicine may require a less arduous evaluation since the previous use of a particular herbal composition as a botanical drug permits clinical trials with multiple chemicals at the outset (i.e., clinical trials using the herbal composition or specific components of the herbal composition). Recently, the FDA has approved the testing of herbal medicines in clinical trials as botanical drugs (FDA Guidance on Botanical Drugs, August 2000). While these events represent a positive development for health care in general, it also raises important issues regarding the formulation, manufacturing and quality control of herbal medicines and dietary supplements. While rigorous clinical trials (multiarmed, placebo controlled, dose escalation, double blind, etc.) are standard for assessing both safety and efficacy, the FDA guidelines for botanical quality control is still being developed. Currently, a combination of chemical marker compounds, chemical fingerprint analysis and bioassay are required along with verification that the product is free of heavy metals, toxins, pesticides, herbicides, fungicides or other man-made pharmacologically active agents. The multitude of relevant biological responses induced by the multiple chemical species in herbs we believe will become increasingly important to support marketing approval by the FDA. Multiple biological response methods can now be employed to monitor the biological activity of a multicomponent or a single molecular entity. These methods include panels of expressed genes, expressed proteins, cytokines, transcription factors, cellular receptors and small molecule metabolomics. It is the balance of the levels of these different entities rather than the amount of any single entity that is believed to be critical to the overall biological viability of the cell or organism. This concept is at the heart of systems or integrative biology and is finding increased use in the study of complex biological problems.

With an increased appreciation in Western countries for their unique pharmaceutical value, there has been a rising interest in methods to better standardize and characterize botanicals. Herbal-based industries are coming under increasing pressure to upgrade their current practices (see, e.g., Angell et al., supra). The need to apply scientific testing to the preparation and administration of herbal medicines and food supplements has been highlighted by several recent reports of toxicity resulting from ingesting herb-based formulations. For example, one patient who took an herbal-based dietary supplement experienced digitalis toxicity (Slifman et al., 1998, N. Engl. J. Med. 339:806-811). It was subsequently determined that the herb ingredient labeled as plantain in the supplement was actually contaminated with Digitalis lanata, an herb known to contain at least 60 cardiac glycosides. In another instance, an herbal preparation was found to be the cause of chronic lead intoxication in a patient (Beigel et al., 1998, N. Engl. J. Med. 339:827-830). This is not a completely unexpected occurrence since contamination of traditional Asian herbal remedies by lead and other heavy metals is well documented (Woolf et al., 1994, Ann. Intern. Med. 121:729-735).

Characterization of Botanicals. It is well known that the genetic identity (e.g., genera, species, cultivar, variety, clone), age of herbal growth, harvest time, the specific plant part utilized, processing method, geographical origin, soil type, weather patterns, type and rate of fertilizer, and other growth factors have a great impact on the particular chemical composition of any particular herb “harvested” from any particular area.

Increasing numbers of various types of tests have been instituted to assure the consistent quality of herbs used in medicine and as dietary supplements, including inspections at the macro- and microscopic levels as well as a variety of chemical analyses. Currently used practices focus on individual endogenous marker substances that are monitored by chromatographic separation and detection by either UV/VIS or more recently, by mass spectrometry. In some cases multiple markers per botanical are used (e.g., 10-12 for ginseng). However, usually only one or two marker compounds per botanical are utilized. In either case, out of the potentially hundreds of phytochemicals in the botanical extract mixture, only a fraction of the available information is utilized. This problem is compounded because it is not generally known if the marker compound(s) is even responsible for the biological action. In the case of the popular botanical taken for mild depression, St. Johns Wort, for example, the original marker compound used traditionally for purity and biological potency (hypericin) does not in-fact correlate with biological effects. It is now believed that a separate molecule (hyperforin) is the bioactive marker (Chatterjeee, SS. Battacharya, S. K., Wonnemann, M., Singer, A., Muller, W. Z., Scwabe, W. (1998) LifeSci.; 63(6), 499-510).

Several different methods are currently used for profiling. High performance liquid chromatography (HPLC) profile using UV/VIS detection of marker molecules in an herbal extract has become one reference standard. Typically, only a single wavelength that maximizes the absorption of the marker compounds of choice is chosen. More advanced methods that monitor simultaneously multiple wavelengths using a diode array detector is becoming more standard. However, there are problems with this approach. Some of these problems include: (1) some of the bioactive molecules may not absorb UV or visible light; (2) UV/VIS detection often cannot distinguish separate different molecular species that may have the same retention time; (3) absorption properties of the various molecular species may not be proportional to the mass of the material that is present; (4) the amount of a chemical is not necessarily proportional to its biological potency; and (5) there may be synergy between individual chemical species that is responsible for the complex biological activity.

Evaporative light scattering is a second type of detector system that is able to monitor molecules based on light scattering of a nebulized stream of analyte molecules. Complimentary in many ways to UV/VIS, it detects a wide variety of small molecule volatile analytes that can be nebulized into the gas phase and detected by light scattering of a polychromatic light beam. Advantages include: (1) removal of background solvent that may interfere with detection; and (2) similar detector response for a wide range of molecular species, i.e. an improved detector of the mass quantity independent of chemical property. One of the disadvantages is that it can only detect molecules less volatile than the solvent in which it is dissolved.

Mass spectrometry (MS) is an analytical method for determining the accurate masses and relative abundances of components of a beam of ionized molecules or molecular fragments produced from a sample placed in a high vacuum. Electrospray or atmospheric pressure ionization (API) MS, allows one to conveniently work in the liquid phase and interface the MS detector with HPLC systems. MS, unlike UV/VIS, is not dependent on the optical density. In practice, MS is used in conjunction with HPLC or capillary electrophoresis (CE): the HPLC separates the chemicals by physicochemical properties and the MS then can be used to detect and aid in the specific molecular identification. Commercial systems are now available which integrate MS and HPLC including UV/VIS and evaporative light scattering detector (ELSD). Mass spectrometry is limited to samples that are gaseous or volatile at low pressure, or that can be so rendered by derivatization.

As can be seen from the discussion above, the selection of only one or two marker components is not adequate to assure standardization and component composition of pharmaceutically active botanical extracts. Recent publications report a greater variation in the quality of herbs by specific suppliers, and the difficulty of providing biological equivalence of herbal extracts. Furthermore, the correlation between safety and efficacy and chemicals in an herb is not well defined in most cases. Recently, in response to complaints from consumer groups and regulatory agencies (Federal Register, February 6, 1997, Volume 62, No. 25, Docket No. 96M-0417, cGMP in Manufacturing, Packing or Holding Dietary Supplements, Proposed Rules), some herbal manufacturers have begun to implement Good Manufacturing Practice (GMP) which requires stringent controls at all levels.

Chemical and spectroscopic methods have been used to characterize the components of herbal medicines and food supplements. For example, three new hederagenin-based acetylated saponins were isolated from the fruits of Gliricidia sepium using these two methods (Kojima et al., 1998, Phytochemistry 48(5):885-888). The botanical sources of Chinese herbal drugs in a number of commercial samples were inferred by comparing the contents of some characteristic constituents which were analyzed with high-performance chromatography (HPLC) or capillary electrophoresis (CE) (Shuenn-Jyi Sheu, 1997, Journal of Food and Drug Analysis 5(4):285-294). For example, the ratio of ephedrine/pseudoephedrine was used as a marker to differentiate Ephedra intermedia from other species; total alkaloid contents were used to distinguish between species of Phellodendron; and the contents of ginsenosides were used to differentiate between species of Panax. However, these methods do not provide a direct measurement of the effect of the various herbs on the molecular, physiological or morphological responses following human treatment with the herbs.

Using gas chromatography-mass spectrometry and atomic-absorption methods, the California Department of Health Sciences, Food and Drug Branch, recently tested Asian medicines obtained from herbal stores for contaminants (R. J. Ko, 1998, N. Engl. J. Med. 339:847). Of the 260 products they tested, at least 83 (32 percent) contained undeclared pharmaceuticals or heavy metals, and 23 had more than one adulterant. Using high-performance liquid chromatography, gas chromatography, and mass spectrometry, a commercially available combination of eight herbs (PC-SPES), was found to contain estrogenic organic compounds (DiPaola et al., 1998, N. Engl. J. Med. 339:785-791). The researchers concluded that PC-SPES has potent estrogenic activity and that prostate cancer patients that took PC-SPES could confound the results of standard therapies and may experience clinically significant adverse effects. Recently, PC-SPES was recalled from the market by the FDA due to quality control issues and the finding that warfarin, a potent prescription only anti-coagulant, was found in many of the batches (www.fda.gov./medwatch/SAFETY/safety02.htm#SPES (Sep. 20, 2002 update). Gas chromatography data was also collected for different samples of the traditional Chinese medicine ‘wei ling xian’ and correlated to the antiinflammatory activity of the samples (Wei et al., Study of chemical pattern recognition as applied to quality assessment of the traditional Chinese medicine “wei ling xian,” Yao Hsuch Pao 26(10): 772-772 (1991)). However, this study did not generate a matrix fingerprint from the data that would allow one to standardize and compare the sample to other samples of the same or a similar herbal composition.

Changes in protein levels have also been used to characterize the effects of herbal compositions or specific components of herbs. For example, the production of granulocyte colony-stimulating factor (G-CSF) from peripheral blood mononuclear cells was found to vary depending on which specific Chinese herb was added to the culture (Yamashiki et al., 1992, J. Clin. Lab. Immunol. 37(2):83-90). Expression of interleukin-1 alpha receptors was markedly up regulated in cultured human epidermal keratinocytes treated with Sho-saiko-to, the most commonly used herbal medicine in Japan (Matsumoto et al., 1997, Jpn. J. Pharmacol. 73(4):333-336). The expression of Fc gamma 11/111 receptors and complement receptor 3 of macrophages were increased by treatment with Toki-shakuyakusan (TSS) (J. C. Cyong, 1997, Nippon Yakurigaku Zasshi 110(Suppl. 1):87-92). Tetrandrine, an alkaloid isolated from a natural Chinese herbal medicine, inhibited signal-induced NF-kappa B activation in rat alveolar macrophages (Chen et al., 1997, Biochem. Biophys. Res. Commun. 231(1):99-102). The herbs Sairei-to, alismatis rhizoma (Japanese name “Takusha”) and hoelen (Japanese name “Bukuryou”) inhibited the synthesis and expression of endothelin-1 in rats with anti-glomerular basement membrane nephritis (Hattori et al., 1997, Nippon Jinzo Gakkai Shi 39(2):121-128).

The increase or decrease in mRNA levels has also been used as an indicator of the effect of various herbs and herbal components. Intraperitoneal injection of Qingyangshen (QYS), a traditional Chinese medicine with antiepileptic properties, and diphenylhydantoin sodium reduced alpha- and beta-tublin mRNAs and hippocampal c-fos mRNA induction during kainic acid-induced chronic seizures in rats (Guo et al., 1993, J. Tradit. Chin. Med. 13(4):281-286; Guo et al., 1995, J. Tradit. Chin. Med. 15(4):292-296; Guo et al., 1996, J. Tradit. Chin. Med. 16(1):48-51). Treatment of cultured human umbilical vein endothelial cells (HUVECs) with the saponin astragaloside IV, a component purified from Astragalus membranaceus, decreased plasminogen activator inhibitor type I (PAI-1) specific mRNA expression and increased tissue-type plasminogen activator (t-PA) specific mRNA (Zhang et al., 1997, J. Vasc. Res. 34(4):273-280). One component isolated from the root of Panax ginseng was found to be a potent inducer of interleukin-8 (IL-8) production by human monocytes and by the human monocytic cell line THP-1, with this induction being accompanied by increased IL-8 mRNA expression (Sonoda et al., 1998, Immunopharmacology 38:287-294).

Recent advances in nucleic acid microarray technology enable massive parallel mining of information on gene expression. This process has been used to study cell cycles, biochemical pathways, genome-wide expression in yeast, cell growth, cellular differentiation, cellular responses to a single chemical compound, and genetic diseases, including the onset and progression of the diseases (M. Schena et al., 1998, TIBTECH 16:301). Because cells respond to the micro-environment changes by changing the expression level of specific genes, the identities of genes expressed in a cell determine what the cell is derived of and what biochemical and regulatory systems are involved, among other things (Brown et al. 1999, Nature genet., 21 (1) supplement:33). Thus, cellular gene expression profiles portray the origin, the present differentiation of the cell, and the cellular responses to external stimulants. No researchers to date, if any, have attempted to apply these new technologies to study the molecular effects of whole herbal treatments and supplements.

Some researchers have attempted to characterize the effects of the major active constituents isolated from selected herbs. For example, treatment of HUVECs with notoginsenoside R1 (NR1), purified from Panax notoginseng, resulted in a dose- and time-dependent increase in TPA synthesis (Zhang et al., 1994, Arteriosclerosis and Thromobosis 14(7):1040-1046). Treatment with NR1 did not change urokinase-type plasminogen activator and PAI-1 antigen synthesis, nor did it effect the deposition of PAI-I in the extracellular matrix. TPA mRNA increased as much as twofold when HUVECs were treated with NR1, whereas expression of PAI-1-specific mRNA was not significantly affected by NR1. Since most studies on P. notoginseng have involved its mixture with other herbs, the researchers noted that it was difficult to assess how their results relate to the situation in vivo when is used therapeutically in humans (Id., at 1045, second column, first paragraph). In addition, since the researchers only studied one major component of the herb, it is not possible to ascertain the molecular effect of the whole herb or the interactions among components of the herb from this study.

Dobashi et al. (1995, Neuroscience Letters 197:235-238) studied the effect of two of the main components of saiko agents, a Chinese herbal drug used to treat nephrotic syndrome, bronchial asthma and chronic rheumatoid arthritis. Administration of SS-d increased plasma adrenocorticotropin (ACTH) levels, proopiomelanocortin mRNA levels in the anterior pituitary and the CRF mRNA level in the rat hypothalamus in a dose dependent manner. In contrast, treatment with SS-a failed to affect the levels of these molecular markers. While this study indicates that administration of SS-d may have an important role in saiko agents-induced CRF release and CRF gene expression in rat hypothalamus, it fails to address the molecular effect of the herbal medication as a whole.

Kojima et al. (1998, Biol. Pharm. Bull. 4:426-428) describe the utilization of differential display of mRNA to isolate and identify genes transcriptionally regulated in mouse liver by sho-saiko-to, an herbal medicine used for treating various inflammatory diseases in Japan. These researchers limited their study to the use of mRNA differential display techniques in investigating the molecular mechanisms of herbal medicine. It also failed to address effects in multiple organs of treated animals and did not provide any guidance for quality control, new use, and standardization of effects.

Ma Ji et al. (1998, Chinese Medical Journal 111(1):17-23) investigated the therapeutic effect of the herb Astragali membranaceus on sodium and water retention in rats experiencing aortocaval fistula-caused experimental congestive heart failure. Chronic heart failure rats with and without Astraglia treatment were compared for changes in various morphological characteristics (e.g., body weight, serum sodium concentration); physiological characteristics (e.g., mean arterial pressure, heart rate, hematocrit and plasma osmolality); mRNA expression levels (e.g., hypothalamic arginine vasopressin (AVP), AVP V₁ a receptor, renal AVP V₂ receptor, aquaporin-2 (AXP2)) and protein excretion (e.g., plasma atrial monophosphate peptide (ANP) and urinary cyclic guanidino monophosphate (cGMP)). The researchers found that treatment with Astraglia improved cardiac and renal functions, partially corrected abnormal mRNA expressions of the AVP system and AQP2, and improved the renal reaction to ANP. This study did not address using the collected data to guide the development of new formulations or for elucidating the synergistic or other interactions among various herbs in a formula, or validate the differential power of the effects for quality control purposes.

Mathematical and Statistical Evaluation of Botanical Extracts. The concept of determining a numerical measure of similarity between two objects composed of a common set of parameters is frequently used over a diverse set of disciplines, such as Psychology, Biogeography, Chemistry and information theory. A wide variety of similarity measures exist that vary in utility and complexity. The most straightforward measure of similarity is the Euclidean distance between two vectors with a Euclidean metric. For a review of similarity measures in the context of chemical substructures see Willett et al., “Chemical Similarity Searching,” J. Chem. Info. Comput. Sci., Vol. 38, pp. 983-996 (1998).

Numerical indicators have been developed in various industries, particularly in the food science industry, to determine a quantitative measure of sample quality, typically referred to as a “quality index.” The quality index can be derived as a function of tens to hundreds of biological and physiochemical parameters. For example, wines are characterized by an aromatic index derived from gas chromatography mass spectral peak concentrations of marker compounds for wines of different vintages (Falque et al., “Differentiation of white wines by their aromatic index,” Talanta, Vol. 54, pages 271-281 (2001)) and by clustering wines for a variety of physiochemical parameters (Nogucira et al., “Analytical Characterization of Madeira Wine,” J. Agric. Food Chem.). Recently, a quality index composed of a linear combination of sample pH and concentrations of marker compounds has been derived to measure the freshness of cold-smoked salmon (Jorgensen et al., “Multiple Compound Quality Index for Cold-Smoked Salmon (Salmo Salar) Developed by Multivariate Regression of Biogenic Amines and PIP,” J. Agric. Food. Chem., Vol. 48, pp. 2448-2452 (2000)) and a quality index for Sardine freshness was based on nucleotide degradation in the sample (Vazquez-Ortiz et al., “Application of the Freshness Quality Index (K Value for Fresh Fish to Canned Sardines from Northwestern Mexico,” J. Food Comp. Anal., Vol. 10, pp. 158-165 (1997)). The deterioration of apple juice quality was quantified with an index derived from fluorescent light emission and the absorbance levels of chemicals associated with apple browning (Cohen et al., “A Rapid Method To Monitor Quality of Apple Juice During Thermal Processing,” Lebnsm-Wiss. U.-Technol., Vol. 31, pp. 612-616 (1998)). Instant coffee was analyzed by proton NMR and categorized by principal components analysis and linear discriminant analysis to categorize the samples by manufacturer and coffee type (Charlton, A J et. al., “Application (1)h NMR and multivariate statistics for screening complex mixtures: quality control and authenticity of instant coffee”, J. Agric. Food Chem., 50(11), pp3098-3103 (2002)). A more statistical version of the quality index, based on the Tanimoto coefficient, was formulated to measure the differences between species of Eucalyptus from gas chromatograms (Dunlop et al., “Chemometric analysis of gas chromatographic data of oils from Eucalyptus species,” Chemometrics and Intelligent Laboratory Systems, Vol. 30, pp. 59-67 (1995)). Quality indices for testing air and water contamination have been standardized by the environmental protection agency (EPA) (Office Of Water, U.S. Environmental Protection Agency, “Total Maximum Daily Load Program: National Overview,” Mar. 16, 2000; http://www.epa.gov/OWOW/TMDL/status.html: U.S. Environmental Protection Agency, “Revised Requirements for Designation of Equivalent Methods for PM_{2.5} and Ambient Air Quality Surveillance for Particulate Matter; Final Rule,” Part IV, Jul. 18, 1997).

In the food and plant sciences, most statistical measures of quality and sample variety are based on product classification. The most common classification algorithms, used over a wide variety of contexts, are neural networks (Garcia et al., “Sherry wine vinegars: phenolic composition during aging,” Food Research International, Vol. 32, pp. 433-440 (1999); Moshou et. al., “A neural network based plant classifier,” Computers and Electronics in Agriculture, Vol. 31, pp. 5-16 (2001); Martin et al., “Discrimination between arabica and robusta green coffee varieties according to their chemical composition,” Talanta, Vol. 46, pp. 1259-1264 (1998); “Application of pattern recognition to the discrimination of roasted coffees,” Analytica Chimica Acta, Vol. 320, pp. 191-197 (1996); “Classification of tea samples by their chemical composition using discriminate analysis,” Vol. 43, pp. 415-419 (1996)), and general multivariate statistics such as linear discrimination analysis (Moshou et. al., “A neural network based plant classifier,” Computers and Electronics in Agriculture, Vol. 31, pp. 5-16 (2001)) and principal component analysis (PCA) (Goodner et al., “Orange, Mandarin, and Hybrid Classification Using Multivariate Statistics Based on Carotenoid Profiles,” J. Agric. Food Chem., Vol. 49, pp. 1146-1150 (2001)). In all cases, the quality index and classification algorithms are based on the a priori selection of a set of individual marker compounds as descriptors and do not take into consideration the balance or ratio of compounds within the overall chemical pattern or comprehensive biological response.

As shown by the above review of relevant scientific articles, powerful statistical and computational methods have not been used to test and standardize botanical extracts containing multiple components, such as herbal compositions, nor have they been used in the improvement and development of therapeutics utilizing biological extracts. The therapeutic value of botanicals is inherent in the multi-component nature of the formulated extracts working synergistically on multiple biological pathways within the human body. Thus, a requirement for effective biological action is not only the individual phytochemical components, but also the balance of ratios of these different components. To understand how these mixtures work and to comprehensively assess the nature of the phytochemical mixtures, it is critical to assess the entire pattern of chemical species and to utilize a variety of both high resolution chemical detectors as well as biological assays that serve effectively as biological detectors. This invention embodies the concept of how to incorporate complete patterns of chemical and biological fingerprints into a single complex matrix and transform this matrix into a small number of values for quantitative comparisons and assessments.

### SUMMARY OF THE INVENTION

The present invention provides computational methodologies necessary to guide the standardization of herbal compositions; to determine which specific components of herbal compositions are responsible for particular biological activities; to predict the biological activities of herbal compositions; for the development of improved herbal therapeutics; for adjusting or modifying an herbal composition; for measuring the relatedness of different herbal compositions; for identifying specific molecules in the batch herbal composition which retain the desired biological activity; for determining which herbal components of a known herbal composition can be eliminated from the known herbal composition while maintaining or improving the desired biological activity of the known herbal composition; for identifying new uses and previously unknown biological activities for the batch herbal composition; and for using the predicted biological activity of the batch herbal composition to aid in the design of therapeutics which include herbal components and synthetic chemical drugs, including the design of therapeutics using the methods of combinatorial chemistry.

The methodologies focus on utilizing all available chemical data that can be collected from high resolution analytical methods including chromatography coupled with UV/VIS, MS, NMR, Raman, IR, etc., digitizing the data, and converting the digital data into a matrix pattern that can be analyzed by different mathematical and/or statistical methods. This method can be extended to also incorporate digital data from biological detectors, including genomics, proteomics, enzyme/receptor arrays, cellular assays, animal assays, and clinical data. The biological data can then be used in two general ways. First, it can be combined directly with the chemical data to create a merged comprehensive matrix fingerprint. Secondly, the biological data can be used to filter the matrix fingerprint generated from the chemical data to define a bio-relevant sub-set. Using this approach, all or a sub-set of data can be utilized, no a priori knowledge of marker compounds is required, and patterns and analysis are defined both by the chemical and bioresponse results, as well as the ratios of chemical and bioresponse results. The key to this approach is the utilization of full matrix patterns of multiple chemical and biological readouts.

### BRIEF DESCRIPTION OF THE FIGURES AND TABLES

FIG. 1. A representative three-dimensional plot of LC-MS (i.e., liquid chromatography-mass spec) data illustrating the signature landscape profile of a botanical multicomponent extract. Retention time (in minutes) on a C18 column is plotted along one dimension, high resolution mass (in atomic mass units) is plotted along a second dimension and the MS intensity (log (Ion Count)) is plotted in the third dimension. The two dimensional trace in the rear of the plot is the UV/VIS absorbtion profile. Note that a single UV/VIS peak may well include multiple unique masses associated with a different, unique molecule in the mixture. It is the peak heights and the ratio of the peak heights that define the ruggedness of the landscape that can be digitized, indexed and encoded into the matrix for further analysis.

FIG. 2. Illustration of the matrix format (M) with datapoint intensities (Iₙ) along the diagonal and individual intensity ratios (Iₘ/Iₙ) placed in the off diagonal. Only one half of the off-diagonal peaks need to be utilized. The off-diagonal intensity ratio between all pairs of datapoints encodes for important synergies or interaction relationships between those datapoints. The relationship between datapoints is lost by focusing solely on individual datapoint intensities. The matrix method can conceptually be extended to higher dimensions by examining other data intrarelationship information. We have used for illustration purposes only a two dimensional matrix for clarity.

FIG. 3. SELDI/TOF (Ciphergen®) spectra of proteins captured on IMAC surface chips that are expressed in Jurkat cells after 24 hour bioresponse treatment with the botanical formulation PHY906 at four different doses (0.0, 0.02, 0.10, 1.0 mg/ml) from top to bottom. There are multiple qualitative changes between the different spectra in the molecular weight range between 5000 and 20,000. These data can be digitized, indexed and encoded into the matrix for further analysis.

FIG. 4(A). Conventional linear correlation (LSQ from software SPLUS) comparing individual peaks between two batches (Scute1 and Scute2) of Scutellaria Radix, i.e. a linear correlation of the diagonal only of the matrix. The dotted line indicates the 95% confidence level. The correlation coefficient for this linear fit is 0.95. However, most of the datapoints are clustered with low intensity and hence are difficult to judge the outliers. (B). Conventional linear correlation comparing individual peaks between two batches (Scute8 and Scute9) of Scutellaria Radix, i.e. a linear correlation of the diagonal only of the matrix. The dotted line indicates the 95% confidence level. The correlation coefficient for this linear fit is 0.995, significantly better than that observed in FIG. 4A but still indicating potential outliers. These datapoints are also used later in calculating a similarity index (Phytomics Similarity Index (PSI); see Equation #7) using the matrix method. See Table 4.

FIG. 5(A). Histogram plot of weighted R-values computed from the intensity ratio matrix for individual datapoints using the same datapoints as in FIG. 4A (Scute1 and Scute2). While the distribution peaks around 0.9, there are clear outliers of individual datapoints less than 0.6. The average of the weighted R-values, defined as PSI (Equation #7) is 0.89. (B). Histogram plot of weighted R-values computed from the intensity ratio matrix for individual datapoints using the same datapoints as in FIG. 4B (Scute8 and Scute9). The distribution peaks around 0.94 and there is only one outlier of an individual datapoint less than 0.6. The average of the weighted R-values, defined as PSI is 0.97. Note that outliers are easier to define and there is a greater numerical spread due to the method by which the R-value is computed, i.e. using the entire ratio set for the particular datapoint, but that the overall comparison is qualitatively similar. Note that the PSI value is computed such that the average value lies between 0.0 and 1.0, where 0.0 is complete dissimilarity and 1.0 is complete identity.

FIG. 6 (A). Histogram plot of unweighted R-values computed from the intensity ratio matrix for individual datapoints (LC/MS peaks) between two batches (Scute5 and Scute6) of the botanical extract Scutellaria Radix. (B). Histogram plot of weighted R-values computed from the intensity ratio matrix for the same datapoints as in FIG. 6A (Sucte5 and Scute6) where the weight is related to a scaling factor involving the original intensities of the datapoint and applied to the correlation R-value of the ratio matrix as defined in Equation # 7 (see Examples). While the unweighted and weighted PSI are the same value (0.97), the distribution of individual datapoint R-values are distributed over a wider range in the weighted PSI, making identification of outliers more reliable.

FIG. 7. Histogram plot of the weighted PSI values from LC/MS data for pairwise comparisons of nine batches of Scutellaria Radix extracts as tabulated in Table 4. A common set of forty-six peaks were used to construct the matrix. The distribution of PSI values clearly demarks a cut-off for these data of approximately 0.95.

FIG. 8. A screenshot of the software PhytoViewer™ used to compute the matrix and PSI values, to display the results and to interrogate the data. The software is written in JAVA and runs on a PC or other computer platforms. In this screenshot, we view the histogram of the matrix correlations for individual datapoints of the LC/MS data of Scute5 and Scute6 of Scutellaria Radix, and illustrate how individual datasets can be selected and merged into a matrix dataset, an interactive histogram view and a query window displaying individual datapoints (LC/MS peaks) from the histogram. In this way, outlier peaks can be immediately identified and queried further.

FIG. 9(A). Histogram plot of weighted PSI values comparing non-treated and post-treated extracts (mimic of digestive process) of nine batches of Scutellaria Radix as in Table 5. Clearly there are two classes of botanical extracts, one strongly susceptible to the post-treatment and one only mildly susceptible. Interrogation of the datapoints (LC/MS peaks of individual compounds) that are highly affected can be used to grade and classify lots of material based on their post-treatment susceptibility. (B). Histogram plot of the difference in weighted PSI value between pairwise untreated and treated batches of Scutellaria Radix (nine batches) indicates that PSI value differences less than 0.2 can be used to classify susceptible from non-susceptible batches.

FIG. 10. A second screenshot of the software PhytoViewer™ used to compute the matrix and PSI values, to display the results and to interrogate the gene expression data. In this screenshot, we view the histogram of the matrix correlations for individual datapoints of the genomics data comparing two separate experiments (SB and SB) selected from the menu on the left hand scroll box and highlighting the genes (accession code) that are in poor agreement between the two experiments. The overall weighted PSI value is 0.91 with the majority of the datapoints (genes) centered around 0.9. This figure illustrates that the same software and methods can be used both for chemical as well as bioresponse data to compare two multicomponent mixtures.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Overview of the Invention

As set forth above, the present invention is directed to software tools and computational methods useful for characterizing and/or predicting the biological response of a biological extract, such as an herbal composition. More particularly, this invention provides methods of creating matrix fingerprints from analytical studies of multicomponent chemical samples such as extracts of botanicals or herbal drugs, and the multifactorial biological effects of such extracts (or single compound). In addition, this invention provides methods as well for using such fingerprints to measure pattern similarity/differences such as different patterns of molecules extracted from different batches of botanicals or differences in the biological response pattern and to use as a guide to assess chemical or biological equivalency and as a guide to improve the design of effective botanical or multicomponent based therapeutics. The goal of the present invention is the overall design, creation, improvement and use of matrix fingerprints for the preparation, testing and administration of herbal compositions, and guiding development of new herbal compositions and novel uses of existing herbal compositions. This method can be applied in cases where (1) the data can be quantitated and digitalized and (2) there are important inter-relationships between the individual data points.

Phytomics. As used herein, depending on the context in which it is used, “phytomics” refers to using bioinformatics and statistical approaches to address the qualitative and quantitative aspects of the components of herbal compositions or to the actual databases that are developed for addressing such aspects.

Matrix Fingerprint. As used herein, the term “matrix fingerprint” as used herein refers to the means of making a characteristic profile of a substance, particularly a botanical extract such as an herbal composition. In order to create a matrix fingerprint, data from chemical and/or biological analyses are digitized and placed along the diagonal of the matrix fingerprint, and ratios of each data point to every other data point is placed in the off-diagonal positions of the matrix. The use of the ratios of the digitized data points at the off-diagonal positions of the matrix fingerprint captures the concept of synergistic interrelationships between the multiple components of the biological extract and their biological actions and defines a pattern landscape that describes the chemical fingerprint of the multicomponent mixture or a multifactorial biological response of the effects of one or more chemical components on a biological system Digitized datapoints for use in the matrix fingerprinting can be obtained using various chemical and biological testing. Examples include, but are not limited to chemical analysis data resulting in resolvable and multiple peaks e.g. LC-MS, MS-MS, GC-MS, electrophoresis, UV-VIS, IR, RAMAN, MALDI, SELDI, ICP-MS and biological analysis data resulting in discrete, digitized data, e.g. genomic microarray, proteomic microarrays, enzyme panels, chemokine panel, receptor panels, metabolite panels where panel is defined as a group of related assays.

Biological Extract/Herb. The terms “Biological extract” and “herb” are used interchangeably throughout this disclosure. Technically speaking an herb is a small, non-woody (i.e., fleshy stemmed), annual or perennial seed-bearing plant in which all the aerial parts die back at the end of each growing season. Herbs are valued for their medicinal, savory or aromatic qualities. As the word is more generally used and as the word is used herein, an “herb” refers to any plant or plant part which has a food supplement, medicinal, drug, therapeutic or life-enhancing use. Thus, as used herein, an herb is not limited to the botanical definition of an herb but rather to any botanical, plant or plant part used for such purposes, including any plant or plant part of any plant species or subspecies of the Metaphyta kingdom, including herbs, shrubs, subshrubs, and trees. Plant parts used in herbal compositions include, but are not limited to, seeds, leaves, stems, twigs, branches, buds, flowers, bulbs, corms, tubers, rhizomes, runners, roots, fruits, cones, berries, cambium and bark.

Herbal Composition. As used herein, an “herbal composition” refers to any composition that includes herbs, herbal plants or herbal plant parts. Thus, as used herein, an herbal composition is any herbal preparation, including herbal food supplements, herbal medicines, herbal drugs and medical foods. Examples of herbal compositions include, but are not limited to, the following components: a whole plant or a plant part of a single plant species; whole plants or plant parts of multiple plant species; multiple components derived from a single plant species; multiple components derived from multiple plant species; or any combination of these various components. For a thorough review of various herbal compositions, see, for example, Kee Chang Huang, The Pharmacology of Chinese Herbs, CRC Press (1993), herein incorporated in its entirety. Representative examples of various herbal compositions are provided in the following paragraphs.

Standardized Herbal Composition. As used herein, a “standardized herbal composition” or a “characterized herbal composition” refers to a particular herbal composition that is chosen as the standard herbal composition for evaluating batch herbal compositions which have the same, similar or different components as the components of the standardized herbal composition. Standardized herbal compositions are generally herbal compositions which have been well characterized and which demonstrate the desired biological responses in a particular biosystem. Standardized herbal compositions are usually standardized by chemical tests well known to one skilled in the art and are properly stored for long term usage and reference. The standardized herbal composition is used to establish a standardized HBR Array based on observations and measurements for the plants (i.e., plant-related data), markers and BioResponses so as to characterize the herbal composition.

Batch Herbal Composition. As used herein, a “batch herbal composition” refers to any test herbal composition that is used to establish a matrix fingerprint based on chemical and biological testing of the biological extract. Sometimes herein also referred to as a “test” herbal composition. Observations and measurements of BioResponses may or may not be included. The herbal compositions used to establish the standardized herbal composition may also be referred to as “batch herbal compositions” until designated as “standardized herbal compositions.”

Batch. As used herein, a “batch” refers to a particular quantity of an herbal composition which can be identified as to some particular attribute so as to distinguish it from any other particular quantity of that same herbal composition. For example, one batch of an herbal composition may differ from another batch of that same herbal composition in that one of the batches was harvested at a different time or in a different geographical location than the other batch. Other differences that distinguish particular batches may include, but are not limited to, the following: 1) the particular plant part used (e.g., the root of an herb was used in one batch while the leaves of that same herb were used in a different batch); 2) the post-harvest treatment of the individual herbs or herbal composition (e.g., one batch may be processed with distilled water while a different batch may be processed with Hydrogen Chloride to simulate the acidity of the human stomach); and, 3) the relative proportions of the individual herbs in an herbal composition (e.g., one batch may have equal parts by weight or volume of three different herbs while another batch has proportionally more of one herb than the other two).

Biosystem. As used herein, a “biosystem” refers to any biological entity for which biological responses may be observed or measured. Thus, a biosystem includes, but is not limited to, any cell, tissue, organ, whole organism or in vitro assay.

Biological Activity. As used herein, the “biological activity” of an herb refers to the specific biological effect peculiar to an herbal composition on a given biosystem.

Chemical Data. Chemical characterization may be accomplished by any chemical analysis method generally known by one skilled in the art. Examples of applicable chemical analyses include, but are not limited to, GC (gas chromatography), HPLC (high pressure liquid chromatography), TLC (thin layer chromatography), electrophoresis, with chemical fingerprinting by one or a combination of UV/VIS, MS, ELSD, IR, NMR or other analysis

Other Plant-Related Data. As used herein, “Plant-related data” refers to the data collected on the herbal composition including, but not limited to, data about the plants, their growing conditions and the handling of the plants during and after harvesting. The plant-related data also includes the relative proportions of the components in an herbal compositions, wherein the components may be different plant parts, different plant species, other non-plant ingredients (e.g., insect parts, chemical drugs) or any combinations of these variables.

Plant-related data which may be gathered for an herbal composition includes, but is not limited to, the following: 1) the plant species (and, if available, the specific plant variety, cultivar, clone, line, etc.) and specific plant parts used in the composition; 2) the geographic origin of the herbs, including the longitude/latitude and elevation; 3) the growth conditions of the herbs, including fertilizer types and amounts, amounts and times of rainfall and irrigation, average microEinsteins received per day, pesticide usage, including herbicides, insecticides, miticides and fungicides, and tillage methods; 4) methods and conditions used for processing the herbs, including age/maturity of the herbs, soaking times, drying times, extraction methods and grinding methods; and 5) storing methods and conditions for the herbal components and the final herbal composition.

Bioinformatics. As used herein, “bioinformatics” refers to the use and organization of information of biological interest. Bioinformatics covers, among other things, the following: (1) data acquisition and analysis; (2) database development; (3) integration and links; and (4) further analysis of the resulting database. Nearly all bioinformatics resources were developed as public domain freeware until the early 1990s, and much is still available free over the Internet. Some companies have developed proprietary databases or analytical software.

Genomic or Genomics. As used herein, the term “genomics” refers to the study of genes and their function. Genomics emphasizes the integration of basic and applied research in comparative gene mapping, molecular cloning, large-scale restriction mapping, and DNA sequencing and computational analysis. Genetic information is extracted using fundamental techniques, such as DNA sequencing, protein sequencing and PCR.

Gene function is determined (1) by analyzing the effects of DNA mutations in genes on normal development and health of the cell, tissue, organ or organism; (2) by analyzing a variety of signals encoded in the DNA sequence; and (3) by studying the proteins produced by a gene or system of related genes.

Proteomic or Proteomics. As used herein, the term “proteomics”, also called “proteome research” or “phenome”, refers to the quantitative protein expression pattern of a genome under defined conditions. As used generally, proteomics refers to methods of high throughput, automated analysis using protein biochemistry.

Conducting proteome research in addition to genome research is necessary for a number of reasons. First, the level of gene expression does not necessarily represent the amount of active protein in a cell. Also, the gene sequence does not describe post-tranlsational modifications, which are essential for the function and activity of a protein. In addition, the genome itself does not describe the dynamic cell processes which alter the protein level either up or down.

Proteome programs seek to characterize all the proteins in a cell, identifying at least part of their amino acid sequence of an isolated protein. In general, the proteins are first separated using 2D gels or HPLC and then the peptides or proteins are sequenced using high throughput mass spectrometry. Using a computer, the output of the mass spectrometry can be analyzed so as to link a gene and the particular protein for which it codes. This overall process is sometimes referred to as “functional genomics”. A number of commercial ventures now offer proteomic services (e.g., Pharmaceutical Proteomics™, The ProteinChip™ System from Ciphergen Biosystem; PerSeptive Biosystems).

For general information on proteome research, see, for example, J. S. Fruton, 1999, Proteins, Enzymes, Genes: The Interplay of Chemistry and Biology, Yale Univ. Pr.; Wilkins et al., 1997, Proteome Research: New Frontiers in Functional Genomics (Principles and Practice), Springer Verlag; A. J. Link, 1999, 2-D Proteome Analysis Protocols (Methods in Molecular Biology 112, Humana Pr.; Kamp et al., 1999, Proteome and Protein Analysis, Springer Verlag.

Signal Transduction. As used herein, “signal transduction”, also known as cellular signal transduction, refers to the pathways through which cells receive external signals and transmit, amplify and direct them internally. Signaling pathways require intercommunicating chains of proteins that transmit the signal in a stepwise fashion. Protein kinases often participate in this cascade of reactions, since many signal transductions involve receiving an extracellular chemical signal, which triggers the phosphorylation of cytoplasmic proteins to amplify the signal.

Post-translational Modification. As used herein, “post-translational modification” is a blanket term used to cover the alterations that happen to a protein after it has been synthesized as a primary polypeptide. Such post-translational modifications include, but are not limited to, glycosylation, removal of the N-terminal methionine (or N-formyl methionine), signal peptide removal, acetylation, formylation, amino acid modifications, internal cleavage of peptide chains to release smaller proteins or peptides, phosphorylation, and modification of methionine.

Array or Microarray. As used herein, an “array” or “microarray” refers to a grid system which has each position or probe cell occupied by a defined nucleic acid fragment. The arrays themselves are sometimes referred to as “chips”, “biochips”, “DNA chips” or “gene chips”. High-density nucleic acid microarrays often have thousands of probe cells in a variety of grid styles.

Once the array is fabricated, DNA or protein molecules derived from a biosystem are added and some form of chemistry occurs between the DNA or protein molecules and the array to give some recognition pattern that is particular to that array and biosystem. Autoradiography of radiolabeled batches is a traditional detection strategy, but other options are available, including fluorescence, colorimetry, and electronic signal transduction.

Data Points As used herein, the term “data points” refer to any chemical- or biological-based measurements that are discrete quantified values used in the calculation of the matrix fingerprint. Such information that would be incorporated into a datapoint include but are not limited to retention times, wavelengths, absorbtion intensity, NMR chemical shift, mass value, mass intensity, gene name/number, protein name/number, gene expression level, protein intensity etc. i.e. any data collected from the mutlicomponent sample or from the multiple biological effects of a single or multicomponent sample from experimental methods or from computed values from such data. The exact identification of the peak (i.e. molecular name/structure, protein or gene name etc.) need not be known as long as data can be associated with each datapoint. Datapoints may also include not only characteristics of the botanical composition but in vitro, cell-based, animal based or human based bioresponse data as described in these various definitions.

The data point database may constitute a data set which enumerates, quantitates and characterizes chemical or biological information.

Marker. As used herein, “Marker” is a single chemical or biological entity that is used as an internal or external reference standard for both calibration and quantification of the experimental data. Examples would include glycyrrhizin and the ginsennosides Rg1, Rb1 as chemical standards for licorice and ginseng botanicals and a variety of housekeeping genes as invariant markers in a microarray. According to the American Botanical Council (Austin, Tex., USA), “A compound whose presence and level are used as an indicator of consistency and quality of a botanical material. A marker compound also may be (but does not need to be) an indicator of identity. Marker compounds may or may not be recognized as having pharmacological activity.” (American Botanical Council, Austin, Tex., USA).

BioResponses. As used herein, a “BioResponse” refers to any observation or measurement of a biological response of a biosystem following exposure to an herbal composition. Sometimes herein also referred to as a “biological effect.” A BioResponse is a qualitative or quantitative data point for the biological activity of a particular herbal composition. BioResponse data includes both dosage and temporal information, wherein such information is well known to one skilled in the art of measuring responses of biosystems to various treatments. Thus, BioResponse data includes information on the specific biological response of a specific biosystem to a specific dosage of herbal composition administered in a particular manner for a specific period of time.

BioResponses include, but are not limited to, physiological responses, morphological responses, cognitive responses, motivational responses, autonomic responses and post-translational modifications, such as signal transduction measurements. Many herbal compositions demonstrate more than one BioResponse (see, e.g., Kee Chang Huang, The Pharmacology of Chinese Herbs, CRC Press (1993)). Some particular BioResponses may be included in more than one of the delineated groups or have aspects or components of the response that encompass more than one group. BioResponses applicable to the instant invention are well known to one skilled in the art. The following references are representative of the state of art in the field: Kee Chang Huang, The Pharmacology of Chinese Herbs, CRC Press (1993); Earl Mindell, Earl Mindell's Herb Bible, Simon & Schuster (1992); Goodman & Gilman's The Pharmacological Basis of Therapeutics, Ninth Edition, Joel G. Hardman, et. al. (eds.), McGraw Hill, Health Professions Division (1996); P. J. Bentley, Elements of pharmacology, A primer on drug action, Cambridge University Press (1981); P. T. Marshall and G. M. Hughes, Physiology of mammals and other vertebrates, Second Edition, Cambridge University Press (1980); Report of the Committee on Infectious Diseases, American Academy of Pediatrics (1991); Knut Schmidt-Nielsen, Animal Physiology: Adaptation and Environment, 5th Edition, Cambridge University Press (1997); Elain N. Marieb, Human Anatomy & Physiology, Addison-Wessley Pub. Co. (1997); William F. Ganong, Review of Medical Physiology (18th Ed), Appleton & Lange (1997); Arthur C. Guyton and John E. Hall, Textbook of Medical Physiology, W. B. Saunders Co. (1995).

A “physiological response” refers to any characteristic related to the physiology, or functioning, of a biosystem. Physiological responses on a cellular, tissue or organ level include, but are not limited to, temperature, blood flow rate, pulse rate, oxygen concentration, bioelectric potential, pH value, cholesterol levels, infection state (e.g., viral, bacterial) and ion flux. Physiological responses on a whole organism basis include gastrointestinal functioning (e.g., ulcers, upset stomach, indigestion, heartburn), reproductive tract functioning (e.g., physiologically-based impotence, uterine cramping, menstrual cramps), excretory functions (e.g., urinary tract problems, kidney ailments, diarrhea, constipation), blood circulation (e.g., hypertension, heart disorders), oxygen consumption, skeletal health (e.g., osteoporosis), condition of the cartilage and connective tissues (e.g., joint pain and inflammation), locomotion, eyesight (e.g., myopia, blindness), muscle tone (e.g. wasting syndrome, muscle strains), presence or absence of pain, epidermal and dermal health (e.g., skin irritation, itching, skin wounds), functioning of the endocrine system, cardiac functioning, nervous coordination, head-related health (e.g., headaches, dizziness), age (e.g., life span, longevity) and respiration (e.g., congestion, respiratory ailments).

A “morphological response” refers to any characteristic related to the morphology, or the form and structure, of a biosystem following exposure to an herbal composition. Morphological responses, regardless of the type of biosystem, include, but are not limited to, size, weight, height, width, color, degree of inflammation, general appearance (e.g., opaqueness, transparency, paleness), degree of wetness or dryness, presence or absence of cancerous growths, and the presence or lack of parasites or pests (e.g., mice, lice, fleas). Morphological responses on a whole organism basis include, but are not limited to, the amount and location of hair growth (e.g., hirsutism, baldness), presence or absence of wrinkles, type and degree of nail and skin growth, degree of blot clotting, presence or absence of sores or wounds, and presence or absence of hemorrhoids.

A “cognitive response” refers to any characteristic related to the cognitive, or mental state, of a biosystem following exposure to an herbal composition. Cognitive responses include, but are not limited to, perceiving, recognizing, conceiving, judging, memory, reasoning and imagining.

A “motivational response” refers to any characteristic related to the motivation, or induces action, of a biosystem following exposure to an herbal composition. Motivational responses include, but are not limited to, emotion (e.g., cheerfulness), desire, learned drive, particular physiological needs (e.g., appetite, sexual drive) or similar impulses that act as incitements to action (e.g., stamina, sex drive).

An “autonomic response” refers to any characteristic related to autonomic responses of a biosystem following exposure to an herbal composition. Autonomic responses are related to the autonomic nervous system of the biosystem. Examples of autonomic responses include, but art not limited to, involuntary functioning (e.g., nervousness, panic attacks), or physiological needs (e.g., respiration, cardiac rhythm, hormone release, immune responses, insomnia, narcolepsy).

BioResponses of cells, tissues, organs and whole organisms treated with various herbal compositions or herbal components are well known in the herbal arts. For example, the herbal compositions Sairei-to (TJ-114), alismatis rhizoma (Japanese name ‘Takusha’) and hoelen (Japanese name ‘Bukuryou’) were each found to inhibit the synthesis and expression of endothelin-1 in rats (Hattori et al., Sairei-to may inhibit the synthesis of endothelin-1 in nephritic glomeruli, Nippon Jinzo Gakkai Shi 39(2), 121-128 (1997)). Interleukin (IL)-1 alpha production was significantly promoted by treatment of cultured human epidermal keratinocytes with the herbal medicine Sho-saiko-to (Matsumoto et al., Enhancement of interleukin-1 alpha mediated autocrine growth of cultured human keratinocytes by sho-saiko-to, Jpn J. Pharmacol 73(4), 333-336 (1997). Adding Sho-saiko-to to a culture of peripheral blood mononuclear cells obtained from healthy volunteers resulted in a dose-dependent increase in the production of granulocyte colony-stimulating factor (G-CSF) (Yamashiki et al., Herbal medicine “sho-saiko-to” induces in vitro granulocyte colony-stimulating factor production on peripheral blood mononuclear cells, J Clin Lab Immunol 37(2), 83-90 (1992)). These researchers concluded that the administration of Sho-saiko-to may be useful for the treatment of chronic liver disease, malignant diseases and acute infectious diseases where G-CSF is efficacious. Plasminogen activator inhibitor type I (PAI-1)-specific mRNA expression decreased and tissue-type plasminogen activator (t-PA)-specific mRNA increased after treatment of human umbilical vein endothelial cells (HUVECs) with the saponin astragaloside IV (AS-IV) purified from the Chinese herb Astragalus membranaceus (Zhang et al., Regulation of the fibrinolytic potential of cultured human umbilical vein endothelial cells: astragaloside IV down regulates plasminogen activator inhibitor-I and up regulates tissue-type plasminogen activator expression, J Vasc Res 34(4), 273-280 (1997)). One component out of four components isolated from the roots of Panax ginseng was found to be a potent inducer of IL-8 production by human monocytes and THP-1 cells, and this induction was accompanied by increased IL-8 mRNA expression (Sonoda et al., Stimulation of interleukin-8 production by acidic polysaccharides from the root of panax ginseng, Immunopharmacology 38(3), 287-294 (1998)). By flow cytometric analysis, the expression of Fc gamma 11/111 receptors and complement receptor 3 (CR3) on macrophages were found to be increased by treatment with the Kampo-herbal medicine Toki-shakuyakusan (TSS) (Cyong, New BRM from kampo-herbal medicine, Nippon Yakurigaku Zasshi 110 Suppl 1, 87P-92P (1997)). Using computer image analysis, Chen et al. (Image analysis for intercellular adhesion molecule-1 expression in MRI/1 pr mice: effects of Chinese herb medicine, Chung Hua I Hsuch Tsa Chih 75(4), 204-206 (1995)) found that the distribution intensity of intercellular adhesion molecule-1 (ICAM-1), immunoglobulins and C3 were significantly decreased in MRL/1 pr mice after treatment with the Chinese herb stragalin. Western blot analysis showed that tetradrine, isolated from a natural Chinese herbal medicine, inhibited signal-induced NF-kappa B activation in rat alveolar macrophages (Chen et al., Tetrandrine inhibits signal-induced NF-kappa B activation in rat alveolar macrophages, Biochem Biophys Res Commun 231(1), 99-102 (1997)). Cytogenetic parameters include, but are not limited to, karyotype analyses (e.g., relative chromosome lengths, centromere positions, presence or absence of secondary constrictions), ideograms (i.e., a diagrammatic representation of the karyotype of an organism), the behavior of chromosomes during mitosis and meiosis, chromosome staining and banding patterns, DNA-protein interactions (also known as nuclease protection assays), neutron scattering studies, rolling circles (A. M. Diegelman and E. T. Kool, Nucleic Acids Res 26(13):3235-3241 (1998); Backert et al., Mol. Cell. Biol. 16(11):6285-6294 (1996); Skaliter et al., J. Viol. 70(2):1132-1136 (1996); A. Fire and S. Q. Xu, Proc. Natl. Acad. Sci. USA 92(10):4641-4645 (1995)), and autoradiography of whole nuclei following incubation with radiolabelled ribonucleotides. Biochemical parameters include, but are not limited to, specific pathway analyses, such as signal transduction, protein synthesis and transport, RNA transcription, cholesterol synthesis and degradation, glucogenesis and glycolysis.

Algorithm. As used herein, an “algorithm” refers to a step-by-step problem-solving procedure, especially an established, recursive computational procedure with a finite number of steps. For general information on algorithms, see, for example, Jerrod H. Zar, Biostatistical Analysis, second edition, Prentice Hall (1984); Robert A. Schowengerdt, Techniques for image processing and classification in remote sensing, Academic Press (1983); Steven Gold et al., New Algorithms for 2D and 3D Point Matching: Pose Estimation and Correspondence, Pattern Recognition, 31(8):1019-1031 (1998); Berc Rustem, Algorithms for Nonlinear Programming and Multiple-Objective Decisions, Wiley-Interscience Series in Systems and Optimization, John Wiley & Sons (1998); Jeffrey H. Kingston, Algorithms and Data Structures: Design, Correctness, Analysis, International Computer Science Series, Addison-Wesley Pub. Co. (1997); Steven S. Skiena, The Algorithm Design Manual, Springer Verlag (1997); and Marcel F. Neuts, Algorithm Probability: A Collection of Problems (Stochastic Modeling), Chapman & Hall (1995). For information more specific to the application of algorithms to genetic-based data, see, for example, Dan Gusfield, Algorithms on Strings, Trees, and Sequences: Computer Science and Computational Biology, Cambridge University Press (1997); Melanie Mitchell, An Introduction to Genetic Algorithms (Complex Adaptive Systems), MIT Press (1996); David E. Goldberg, Genetic Algorithms in Search. Optimization and Machine Learning, Addison-Wessley Pub. Co. (1989); Zbigniew Michalewicz, Genetic Algorithms+Data Structures=Evolution Programs, Springer Verlag (1996); Andre G. Uitterlinden and Jan Vijg, Two-Dimensional DNA Typing: A Parallel Approach to Genome Analysis, Ellis Horwood Series in Molecular Biology, Ellis Horwood Ltd. (1994); and Pierre Baldi and Soren Brunak, Bioinformatics: The Machine Learning Approach (Adaptive Computation and Machine Learning), MIT Press (1998).

Set Operations. As used herein, “set operations” refer to the mathematical “intersection”, “union” and “difference” operations on a data set, where each member of the data set is labeled by a classifier. For example, LC-MS data points are composed of a list of peaks where each peak has a measured intensity and is classified by a LC retention time and accurate mass coordinate. Similarly, genomic data points are composed of a list of intensities, each specified by a unique gene identification label. The intersection of two LC-MS data sets therefore is simply the set of peaks with the same binned, time and mass. For genomic data, the intersection operation returns the set of data points with the same gene identification label. The union of two data sets is the set of all distinguishable data points and the difference is the set of all data points unique to both data sets.

Statistical Analyses. As used herein, “statistical analyses” refers to any statistical operation documented in the peer refereed statistics literature. Most statistical methods mentioned here are presented in detail in R. A Johnson, D. A. Wichern, and D. W. Wichern, Applied Multivariate Statistical Analysis, Prentice Hall (1983). The terms “linear correlation” and “Pearson coefficient”, designated here by the symbol R, refer to the calculation of the Pearson correlation coefficient between two data sets.

If we replace the values of each data point by its rank among all the other data points in the data set, we are able to determine the Spearman rank correlation coefficient. The formula for the Spearman rank correlation coefficient is identical to that of the Pearson coefficient except that the values of the data points are replaced by their respective ranks. The advantage of this analysis is that a numerical measure of the coefficients' significance as compared to a null hypothesis can be determined, see E. L Lehmann, Nonparametrics: Statistical Methods Based on Ranks, San Francisco: Holden-Day (1975).

Combinatorial Chemistry. As used herein, “combinatorial chemistry” refers to the numerous technologies used to create hundreds or thousands of chemical compounds, wherein each of the chemical compounds differ for one or more features, such as their shape, charge, and/or hydrophobic characteristics. Combinatorial chemistry can be utilized to generate compounds that are chemical variations of herbs or herbal components. Such compounds can be evaluated using the methods of the present invention.

Basic combinatorial chemistry concepts are well known to one of ordinary skill in the chemical arts and can also be found in Nicholas K. Terrett, Combinatorial Chemistry (Oxford Chemistry, Masters), Oxford Univ. Press (1998); Anthony W. Czarnik and Sheila Hobbs Dewitt (Editors), A Practical Guide to Combinatorial Chemistry, Amer. Chemical Society (1997); Stephen R. Wilson (Editor) and Anthony W. Czarnik (Contributor), Combinatorial Chemistry: Synthesis and Application, John Wiley & Sons (1997); Eric M. Gordon and James F. Kerwin (Editors), Combinatorial Chemistry and Molecular Diversity in Drug Discovery, Wiley-Liss (1998); Shmuel Cabilly (Editor), Combinatorial Peptide Library Protocols (Methods in Molecular Biology), Human Press (1997); John P. Devlin, High Throughput Screening, Marcel Dekker (1998); Larry Gold and Joseph Alper, Keeping pace with genomics through combinatorial chemistry, Nature Biotechnology 15, 297 (1997); Aris Persidis, Combinatorial chemistry, Nature Biotechnology 16, 691-693 (1998).

### EXAMPLES

### Example 1

### Generating a Matrix Fingerprint Using Chemical Data

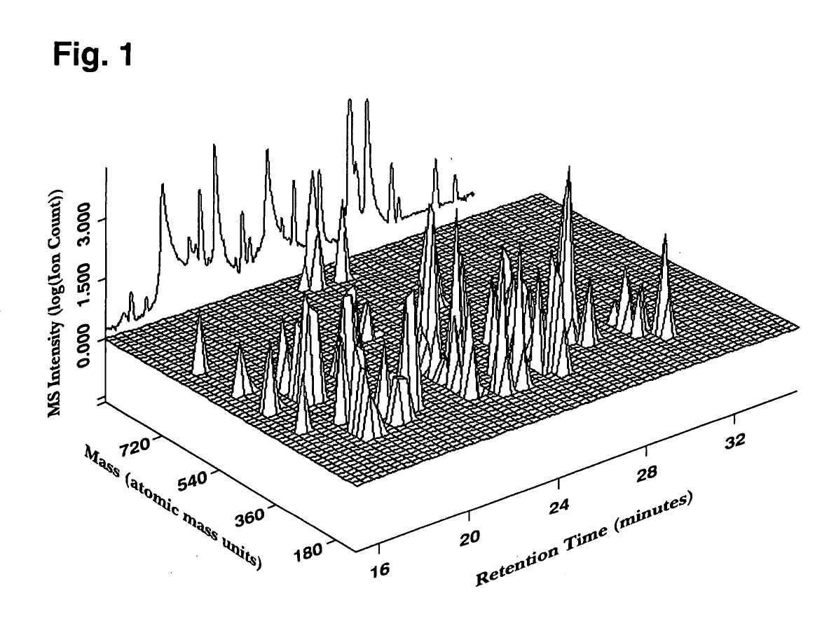

A unique one dimensional, two dimensional, or higher dimensional chemical fingerprint of a multi-component botanical drug can be collected via a number of experimental analytical assays. Detection methods may include UV/VIS, ELSD, infrared, NMR, refractive index, mass spectrometry etc. Any detection method can be used as long as the data generated can be indexed and digitized. We illustrate the generation of a matrix fingerprint with high resolution data from LC-MS of a complex botanical formulation consisting of four botanicals. FIG. 1 shows a small region of a three-dimensional plot of the Liquid Chromatography-Mass Spectrometry (LC-MS) chemical fingerprint for a botanical formulation. Along one dimension of the plot is the separation of individual components along a chromatography separation axis with a noted retention time that can be correlated with a water/octonol partition coefficient (logP) or a computed logP from a unique structure identification. Along a mass spectral axis is illustrated the unique mass of individual chemical components within the multicomponent mixture. As shown in FIG. 1, the third dimension illustrates the intensity of the peak proportional to the number of molecules measured for each of the chemical components.

Multiple compounds can be separated cleanly and the data points generated can be digitized as shown in Table 1 (below). Each datapoint (peak) corresponding in this case to an individual molecule, therefore has three coordinates (retention time (or calculated logP), mass, signal intensity).

TABLE 1
A subset of representative data extracted (retention time,
mass, intensity) or computed (clogP) from spectra such as in
FIG. 1, indexed and used as input for the matrix method. Units
include minutes (retention time) and atomic mass units (mass).

| | Retention Time | | | |
|---|---|---|---|---|
| Peak Number | (min) | clogP | Mass (amu) | Intensity |
| 58 | 13.31 | 0.75 | 419.1316 | 5356 |
| 299 | 17.8 | 0.96 | 461.1077 | 126700 |
| 348 | 18.35 | 1.21 | 461.1074 | 215464 |
| 510 | 22.12 | 2.84 | 823.4122 | 44575 |
| 374 | 19.75 | 2.93 | 271.0591 | 8263 |
| 408 | 20.25 | 3 | 271.0579 | 198204 |
| 527 | 23.13 | 3.08 | 285.0733 | 150195 |
| 453 | 21.14 | 3.11 | 257.079 | 1036 |
| 591 | 23.88 | 3.33 | 285.0723 | 45016 |
| 551 | 23.53 | 3.56 | 255.062 | 7476 |

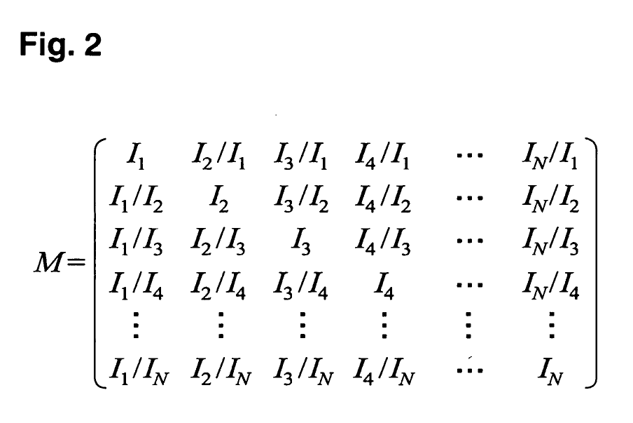

Given a list of N, LC-MS peaks that represent a particular botanical, such as those seen in Table 1, we are able to calculate a full matrix of the intensities of each datapoint peak along the diagonal and equally important the ratio of the intensities of each peak with every other peak in the off-diagonal portion of the matrix as illustrated in FIG. 2.

While it is desirable to have an analytical response for individual molecules, it is not a requirement of the matrix method. For example, the integrated intensity of a UV/VIS peak at a particular retention time is perfectly acceptable in the matrix method, even though more than one compound may be responsible for the UV/VIS intensity (see FIG. 1). The off-diagonal peaks code for the importance of the synergistic balance of the various single chemical components. It is believed that it is not just the intensity of any single peak that is important to the quality control and biological function but that it is the balance of the peaks that confer the overall benefit and biological activity. These ratios are stored in a matrix fingerprint, wherein the matrix fingerprint allows for a variety of mathematical manipulations. Clearly, there are N(N-1)/2 unique non-diagonal elements in the above matrix that are used and would be required to be stored for subsequent calculations. The concepts of computing the full matrix of ratios of the data points and utilizing a matrix to encode and describe the data is a key component of this invention.

### Example 2

### Generating a Matrix Fingerprint Using Biological Data

Both single molecules and multicomponent mixtures of molecules can elicit a multitude of biological responses either in vivo, cell culture or in vitro across a panel of individual biomolecular assays. Very often there are linkages or pattern relationships between individual components of the overall biological response, e.g. one protein level may go up and is counterbalanced by two other protein levels that go down. Other examples would include correlated changes in individual message RNA levels, individual protein expression levels, bioresponse levels of endogenous metabolites, cytokine responses, enzyme activities, cellular pathways etc. We illustrate the construction of a bioresponse matrix from a multicomponent mixture using both genomic and proteomic data as examples.

Genomic Response Fingerprint:

Genomic bioresponse data can be collected by a variety of methods. The most holistic method would include utilizing a microarray or chip technology to measure mRNA levels expressing for individual genes for all known gene sequences. Currently, the state-of-art is upwards of ˜35,000 gene features. The rapid development of nucleic acid microarray technology has led to an explosion of gene expression data (Eisen et. al., (1998), Golub et. al., (1999), Schena M., Shalon D., Davis R. W., and Brown P. O. (1995) Quantitative monitoring of gene expression patterns with a complementary DNA microarray. Science 270:467-470, Eisen M. B., Spellman P. T., Brown P. O., and Botstein D. (1998) Cluster analysis and display of genome-wide expression patterns. Proc. Natl. Acad. Sci. USA 95:14863-14868, Perou C. M., Jeffrey S. S., van de Rijn M., Rees C. A., Eisen M. B., Ross D. T., Pergamenschikov A., Williams C. F., Zhu S. X., Lee J. C., Lashkari D., Shalon D., Brown P. O., and Botstein D. (1999) Distinctive gene expression patterns in human mammary epithelial cells and breast cancers. Proc. Natl. Acad. Sci. USA 96:9212-9217, Tamayo P., Slonim D., Mesirov J., Zhu Q., Kitareewan S., Dmitrovsky E., Lander E. S., and Golub T. R. (1999) Interpreting patterns of gene expression with self-organizing maps: Methods and application to hematopoietic differentiation. Proc. Natl. Acad. Sci. USA 96:2907-2912, Golub T. R., Slonim D. K., Tamayo P., Huard C., Gaasenbeek M., Mesirov J. P., Coller H., Loh M. L., Downing J. R., Caligiuri M. A., Bloomfield C. D., and Lander E. S. (1999) Molecular classification of cancer: class discovery and class prediction by gene expression monitoring. Science 286:531-537, and Ramaswamy S., Tamayo P., Rifkin R., Mukherjee S., Yeang C. H., Angelo M., Ladd C., Reich M., Latulippe E., Mesirov J. P., Poggio T., Gerald W., Loda M., Lander E. S., and Golub T. R. (2001) Multiclass cancer diagnosis using tumor gene expression signatures. Proc. Natl. Acad. Sci. USA 98:15149-15154).

Four characteristics of the gene expression account for the great value of using nucleic acid microarrays to study the gene expression profiles: (i) nucleic acid microarrays makes it easier to measure the transcripts of thousands of genes at once; (ii) close association between the function of a gene product and its expression pattern makes gene function predictable; (iii) cells respond to the micro-environmental changes by changing the expression level of specific genes; and (iv) the sets of genes expressed in a cell determine what the cell is derived of, what biochemical and regulatory systems are involved, and so on (Tamayo et. al., 1999; Ramaswamy et. al., 2001). By using a microarray system, the above features can be studied in an ensemble manner. The expression of any desired number of genes can be detected using the nucleic acid microarray technology. For example, current technology allows up to about 25,000 genes to be placed on a single array. In addition, one can use real time quantitative PCR (RT-qPCR) methods on a selection of the genes to provide higher quality data. Other methods for identifying levels of expressed genes will undoubtedly be defined in the future. In any case, these data are collected for both a treated and baseline system to assess the relative comparison of genes whose expression levels have been altered. Genes are defined into different categories: genes that are induced (up-regulated, higher expression), repressed (down regulated, lower expression), genes that are expressed but unregulated or unchanged, and genes that are not expressed. A list of unique identification numbers along with the relative intensity compared to a control (corrected log ratio), of mRNA coding for the gene is shown in Table 2.

TABLE 2
A subset of typical data from a genomics chip experiment
indicating the individual gene name (as referenced by the
Genbank accession number as described in the website
<http://www.ncbi.nlm.nih.gov/entrez/query.fcgi?db=
Nucleotide> and the corrected log ratio data between
treated and control samples (in this case Jurkat cells treated
with a single PHY906 dose) that can then be indexed and
used as input for the matrix method.

| Peak Number | Gene Name | Corrected Log Ratio |
|---|---|---|
| 1 | 201266_at | 0.4 |
| 2 | 200881_s_at | −0.3 |
| 3 | 204286_s_at | 0.8 |
| 4 | 200779_at | 0.6 |
| 5 | 203474_at | −0.5 |
| 6 | 201690_s_at | 0.6 |
| 7 | 214390_s_at | 0.4 |
| 8 | 219014_at | −1 |
| 9 | 202146_at | 1 |
| 10 | 201791_s_at | 0.3 |
| 11 | 212816_s_at | 2.6 |
| 12 | 207076_s_at | 2.8 |
| 13 | 208964_s_at | 0.3 |
| 14 | 209368_at | 0.8 |
| 15 | 207826_s_at | −1 |
| 16 | 200748_s_at | 1.2 |
| 17 | 212501_at | 1.1 |
| 18 | 203814_s_at | 0.4 |
| 19 | 202672_s_at | 1.1 |
| 20 | 201000_at | 0.7 |

These data were collected from a Jurkat cell line treated for a single day with a 3 day IC50 dose of the botanical formulation PHY906 (composed of four botanicals) using Affymetrix™ Chips, originally containing over 18,000 different gene features of which only ˜100 genes are substantially and consistently altered. As in FIG. 2, we are able to calculate a matrix consisting of the corrected log ratio intensities of each of the genes along the diagonal and the ratio of the intensities of each peak with every other peak in the off-diagonal portion of the matrix. These ratios are stored in a matrix fingerprint M(i, j) wherein the matrix fingerprint allows for a variety of mathematical manipulations. This matrix contains not only the relative expression intensities of individual genes that compose the diagonal matrix elements, but equally important, the intensity ratios of all observed or chosen genes, that compose the off diagonal matrix elements. The off-diagonal genes code for the importance of the synergistic balance of the various gene products in maintaining life processes within the cell. It is believed that it is not only the individual gene intensities that are important to monitor biological function but that it is the balance of the collection of genes that confer the overall biological response.

Proteomics

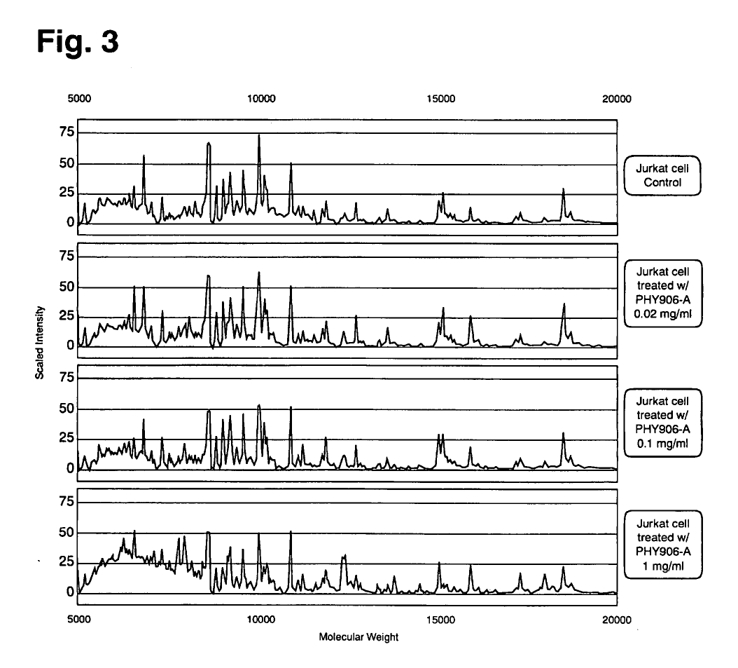

Proteomics is a rapidly evolving set of technologies to identify and quantitate the actual proteins that are coded by the mRNA. In this regard, it is a more direct way to monitor protein levels and to determine the post-translational modifications (phosphorylation, glycosylation etc.) that often modify the functional properties of the protein molecule. Current state-of-art includes 2-d gel electrophoresis and multiple mass spectrometry (MS) methodologies including LC-electrospray MS and MALDI or SELDI MS. In either case, the data can be quantified and indexed allowing for computation of the matrix. We illustrate with data collected on a standard commercially available Protein Chip System™ (Ciphergen BiosystemInc.) using the SELDI method and the metal binding chips (IMAC) Hutchens, T. W., Yip, T. T. (1993, Rapid Comm. Mass Spect. (7), P576.; Fung, E. T., Thulasiraman, V., Weinberger, S. R., Delmaso, E. A., (2001), Curr. Opion. Biotech, (12), p65. In this experiment Jurkat cells treated with a botanical formulation PHY906 are processed to isolate the spectrum of proteins. These proteins are applied to the surface coating of the chip that selectively adsorbs proteins with an affinity for metal binding. This chip is then analyzed by the MALDI-TOF instrument producing a mass spectrum of a sub-set of the expressed proteins that bind to the chip surface. Typical examples of the TOF-MS spectra are shown in FIG. 3 for Jurkat cells treated with different doses of the botanical extract PHY906.

These data are processed with the Ciphergen software, producing a list of peaks, peak number, mass and background and internal standard corrected intensities as shown in Table 3. These data can then be constructed into a matrix as in FIG. 2 with the corrected peak intensity along the diagonal and the ratio of the peak intensities placed in the appropriate off-diagonal positions in an analogous way to the LC/MS data.

TABLE 3
A subset of representative data extracted (mass and corrected
intensity) from SELDI/MS data from a proteomics experiment
(in this case Jurkat cells treated with different doses of PHY906)
from spectra such as in FIG. 3, indexed and used as input
for the matrix method. Units are atomic mass units (mass or amu).

| Peak Number | Protein Mass (amu) | Corrected Intensity |
|---|---|---|
| 1 | 1087 | 32 |
| 2 | 1134 | 21.5 |
| 3 | 1145 | 31.4 |
| 4 | 1185 | 14 |
| 5 | 1333 | 14.5 |
| 6 | 1396 | 17.6 |
| 7 | 3057 | 1.6 |
| 8 | 3307 | 2.4 |
| 9 | 4575 | 6.9 |
| 10 | 5257 | 1.5 |
| 11 | 5552 | 0.7 |
| 12 | 6172 | 5.6 |
| 13 | 6437 | 3.3 |
| 14 | 6541 | 2.2 |
| 15 | 6672 | 6.8 |
| 16 | 8162 | 2.3 |
| 17 | 8451 | 4.4 |
| 18 | 9035 | 2.5 |
| 19 | 9297 | 3.4 |
| 20 | 9398 | 7.5 |

Other Biological Responses

In a similar way any bioresponse data from a panel of assays or observations that can be digitized, indexed and quantified can be incorporated into a matrix format, whereby the response value is placed along the diagonal and relative ratio data between two responses can be placed off the diagonal in the appropriate Mᵢⱼ position. Such biological response data could range from the molecular (e.g., cytokine patterns), biological pathway responses (e.g. signal transduction), transcription factors, isozyme/isoreceptors etc., all the way up to such macro responses as the behavioral level, sleep time, swim times, tail flicks, eating levels etc.

Higher Dimensional Matrix

In principle, the matrix method can be projected into higher (n) dimensions, by examining any number of more complex ratios e.g. (I₁+I₂)/I₃ etc. using a M(i, j, k . . . ) representation. For simplicity, we only focus on two dimensional matrices to illustrate the utility. In addition, while we only focus on pairwise, this method could be extended to perform simultaneous comparisons between multiple sets of data.

### Example 3

### Using Matrix Fingerprints to Compute a Similarity Index Between Samples

When checking for similarity among different botanical samples, one may compare the intensity matrices for each sample instead of matching only the intensities of individual peaks. Since the intensity matrices generated in this way represent all inter-spectrum ratios, the problem at hand is comparing patterns of ratios between two matrices. The statistical correlation of these patterns is a key component embodied in the Phytomics Similarity Index (PSI). We illustrate two examples of the PSI; unweighted and weighted.

The example procedure is as follows: given two samples, first find all the datapoints common in both samples (intersection) and calculate the intensity matrix for each sample using these common datapoints (datapoint for example may represent an LC/MS peak, UV/VIS peak, gene intensity, protein level, cytokine level etc. that has been incorporated into the matrix). Once the matrices are formulated, there exist a wide range of statistical procedures available to compare the patterns of two matrices. One can further perform a large number of known mathematical and statistical operations to analyze and quantify these patterns. The simplest analysis, discussed herein, is the linear correlation of matrix columns between two matrices. To determine the linear correlation, compare all the columns in both matrices A and B, designated M_{A} and M_{B}, ignoring the diagonal components. Each column in matrix A and B is represented by the vectors:
xᵢ^{A}=(Mᵢ₁^{A}, Mᵢ₂^{A}, Mᵢ₃^{A}, Mᵢ₄^{A}, Mᵢ₅^{A}, ΛMᵢⱼ^{A}, ΛM_{iJ}^{A}|i≠j)
xᵢ^{B}=(Mᵢ₁^{B}, Mᵢ₂^{B}, Mᵢ₃^{B}, Mᵢ₄^{B}, Mᵢ₅^{B}, ΛMᵢⱼ^{B}, ΛMᵢⱼ^{B}, ΛM_{iJ}^{B}|i≠j)
where matrix elements with i=j are ignored (Equation # 1).

The correlation strength R for each column, i.e. datapoint, can be obtained with the usual Pearson coefficient,

$$R = \frac{n {\sum }_{}^{} {x}_{A} {x}_{B} - {\sum }_{} {x}_{A} \sum {x}_{B} }{\sqrt{( n {\sum }_{} {x}_{4}^{2} - {( \sum {x}_{A} )}^{2} ) ( n {\sum }^{} {x}_{B}^{2} - {( \sum {x}_{B} )}^{2} )}}$$

or with the Spearman ranked coefficient if one seeks a standardized score (Equation # 2). The result of this analysis is a vector of R scores, where each vector element corresponds to a datapoint (peak, value etc.) common to both datasets. While each datapoint has its own correlation score Rn, one possible definition of the Phytomics Similarity index, or PSI, could be the average of all unweighted R scores to produce a single value. In this example, the R score would range between 0.0 (complete dissimilarity) to 1.0 (complete identity) not unlike the Tanimoto index used to score similarity of chemical fingerprint features.

Since R, as previously defined, only measures the correlation of spectral peaks common to both samples being compared, the PSI score could also be adjusted to account for peaks not present in both spectra. For example, given two LC/MS spectra, A and B corresponding to samples A and B, one such adjustment entails multiplying R by a correlation coefficient, α, defined in terms of the minimum set of peaks present (Equation #3):

$$α = Min ( \frac{A ⋂ B}{A} , \frac{A ⋂ B}{B} ) .$$

Therefore, a corrected, unweighted PSI is then constructed by multiplying the average of the coefficients R by the coefficient α (Equation # 4):

$$PSI = \frac{α}{N} {\sum }_{i = 1}^{N} {R}_{i}$$

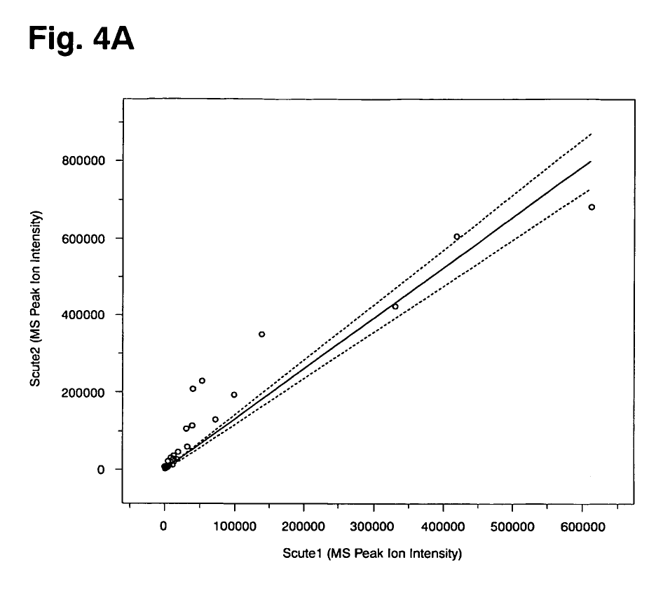

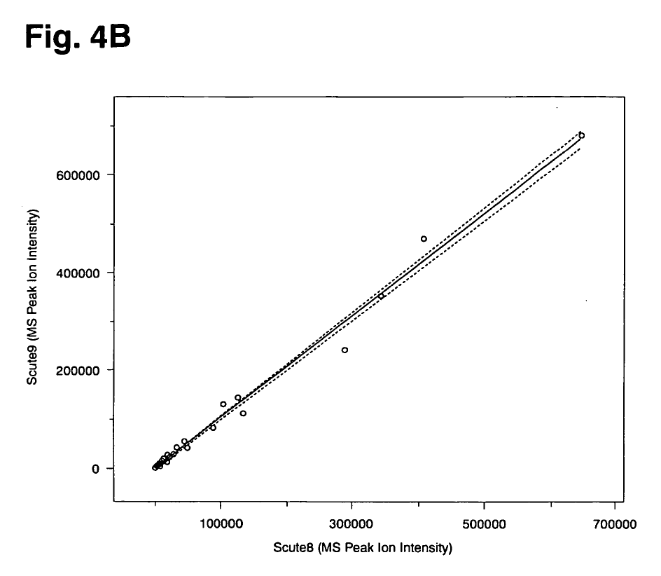

When comparing two spectra, one may simply take the intersection of peaks in both spectra and study the linear correlation of their intensities or perform popular statistical analyses such as PCA or LDA. This is currently the state-of-the-art that is done today, and while it provides a measure of the overall correlation between spectra, it does not provide any measurement of the relationship between the peaks within or between samples. The result of eliminating this information is that any trend or pattern among the same spectral peaks is missed. This qualitative deficiency in current methods is illustrated in FIG. 4 showing a plot of the intensities of peaks common to different batches of the same botanical.

While the overall linear correlation is clear, illustrating the similarity of the two botanical batches, it unfortunately makes it very difficult to detect any pattern among the points since most of the peaks cluster around the region of low intensity. Furthermore, it can be difficult to determine in many cases which peaks should be considered as outliers.

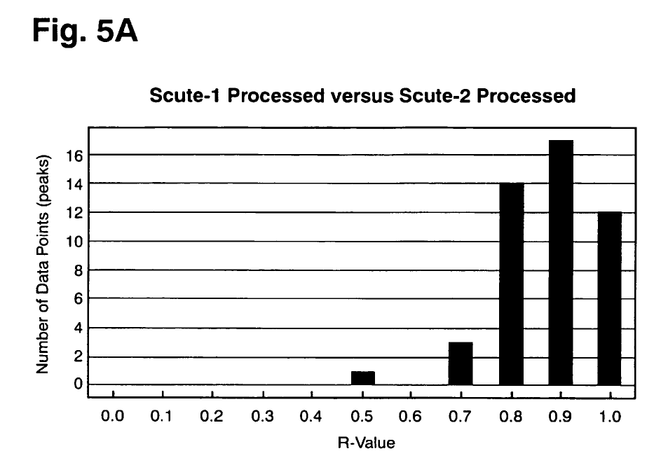

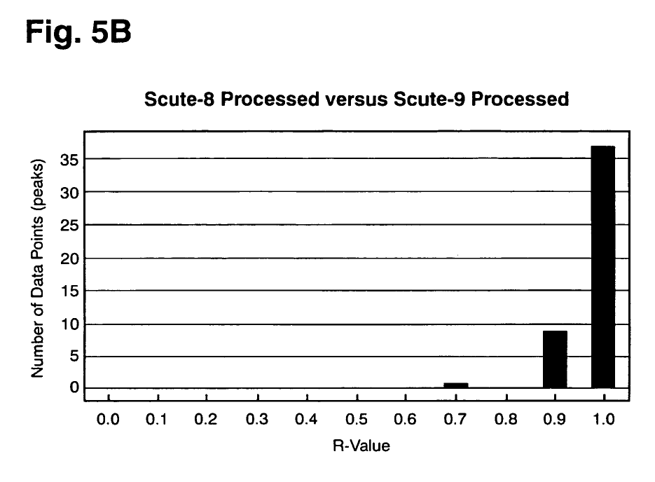

These deficiencies are easily corrected when one includes the intensity ratio matrix method. FIG. 5 shows the distribution of R scores of the ratio sets for individual datapoints when comparing the intensity matrices between batches B1 and B2 and B8 and B9 of a single botanical, Scutellaria Radix.

The distribution for batches B1 and B2, although peaked around 0.9, has several obvious outlier peaks that are related to a small number of underrepresented compounds within the botanical extract. In contrast, batches B8 and B9 have very few outlier peaks, indicating that these batches are better correlated. Clearly, when comparing the results of FIGS. 4 and 5, the correlation of the ratio matrices provides a more powerful tool to determine outlier peaks that can help to establish more accurate specifications for quality control. The ratio comparison tends to reinforce the differences as well as to incorporate the importance of intra-ratio differences.

The matrix correlation method can be extended and generalized to weight the individual terms depending on other information, e.g. confidence in result, importance of data etc. One example of a matrix-weighted correlation (weighted PSI) is to weight the coefficients by the simple linear correlation of the LC-MS intensity information of peaks along the diagonal of the matrix. The matrix correlation method becomes even more powerful if we also use the simple linear correlation as demonstrated in FIG. 4. This information can then be used to weight the distribution of Pearson (or Spearman) coefficients determined from the matrix method. For example, suppose that the slope of the fitted line in FIG. 4 is given by b such that:
Iᵢ^{A}=bIᵢ^{B}+ε₁,
where I^{A} and I^{B} are the intensities of peak i for samples A and B, and ε₁ are the residuals (Equation # 5). We define the weights w, for comparing matrix A versus matrix B, as:

$${w}_{i} = 1 - {( \frac{{b}_{i} - b}{{b}_{i} + b} )}^{2} ,$$

where bᵢ=Iᵢ^{A}|Iᵢ^{B}. Each Pearson coefficient R₁ is weighted by wᵢ (Equation # 6). A second definition and preferred definition of the weighted Phytomics Similarity Index (PSI) is therefore defined as (Equation # 7):

$$PSI = α \frac{{\sum }_{i = 1}^{N} {R}_{i} {w}_{i}}{{\sum }_{i = 1}^{N} {w}_{i}} ,$$

where α is as defined above.

The PSI value computation is only one of many treatments of the matrix data and is used in illustration due to its simplicity of generating a single number for comparison.

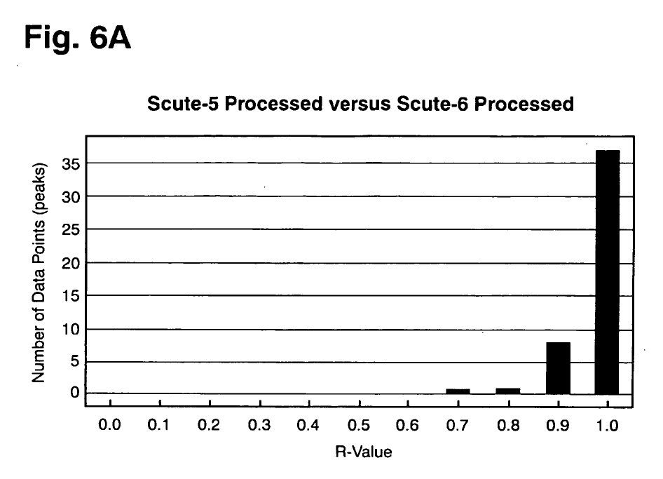

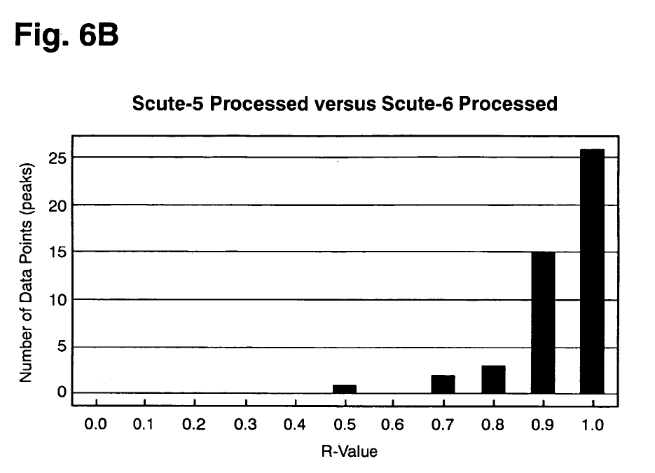

In FIG. 6A the Pearson distribution for typical samples Scute5 and Scute6 are plotted. Also plotted in FIG. 6B is the ‘weighted’ Pearson distribution, wᵢRᵢ.

As shown, the weighted distribution is stretched over a larger range, thus moving outlier points that not well correlated (linearly) closer to zero. In this way, any peaks that correlate well within the matrix correlation are poorly correlated linearly, can be easily identified as outliers. Furthermore, since the overall PSI value is weighted here, it is expected to be less sensitive to the outlier, poorly correlated peaks.

Comparison of the Matrix Method with Traditional Methods

Having formulated a novel methodology to assess the similarity between two herbal compositions, it is important to demonstrate that a comparison between the traditional linear correlation and the matrix method produces similar qualitative results. Consider again the common set of LCMS peaks lists representing measurements of herbal compositions Scute1 and Scute2, where Sucte1 and Scute2 are two batches of the same botanical (Scutellaria Radix), but purchased from different manufacturers. The intensity measurements of the common peaks in Scute1 and Scute2 have a p-value of 0.074, clearly indicating that they were drawn from the same distribution. A plot of the logarithm of intensities from Scute1 versus Scute2 (FIG. 4), along with the results of a linear least square fit. The linear correlation is about 0.95, illustrating a high level of correlation between Scute1 and Scute2. The largest outliers are visually identified to be the (time, mass) pairs (27.53, 315.01), (21.29, 446.64), (24.28, 313.03), (18.42, 446.64), (20.41, 446.636), and (21.87, 271.09). In FIGS. 5A and 5B, the distribution of correlation coefficients using the weightings methods described above is shown, and has a weighted PSI of 0.89. The peaks with the poorest correlation (wᵢRᵢ<0.5) between Scute1 and Scute2 are the exact set of peaks as listed above. The matrix method in all cases does at least as well as conventional methods but provides better methods of distinguishing outliers and, in more subtle comparisons with strong intra-dependencies between measured datapoints, is superior.

### Example 4

### Uses of the Matrix Fingerprints and the PSI Metric

Comparison of the matrix fingerprints discussed herein can be used for many numerical comparative purposes including, but not limited to, the following: 1) evaluating the similarity of the chemical components between herbal compositions; 2) evaluating the BioResponse of an herbal composition; 3) determining those data points that are most highly correlated with a particular BioResponse of an herbal composition; 4) determining what set(s) of information (i.e., plant-related data, chemical data, and BioResponse data) is/are most correlated with a particular BioResponse of an herbal compost; 5) determining which type of biosystem is best for evaluating the biological activity of an herbal composition; 6) adjusting or changing the components of a herbal composition so that the matrix fingerprint of that herbal composition corresponds to a standardized matrix fingerprint for the same or substantially the same herbal composition; 7) adjusting or changing the components of an herbal composition so that the herbal composition will have the desired biological activity; 8) measuring the similarity of different herbal compositions; 9) creating and updating standardized matrix fingerprint; 10) identifying specific components (e.g., plant parts, proteins, molecules) which retain the desired biological activity of an herbal composition; 11) determining which components of an herbal composition can be eliminated while maintaining or improving the desired biological activity of the herbal composition; 12) identifying one or more previously unknown biological activities for an herbal composition; 13) aiding in the design of therapeutics which include herbal and non-herbal components, such as chemically-synthesized drugs or pharmaceuticals and 14) utilizing the matrix fingerprint as a tool that complements combinatorial chemistry methods of designing therapeutics. Each of these embodiments of the present invention can be accomplished by one skilled in the applicable art using the methods and tools commonly used or provided herein.

### Example 5

### Quality Control (Chemical Fingerprint)

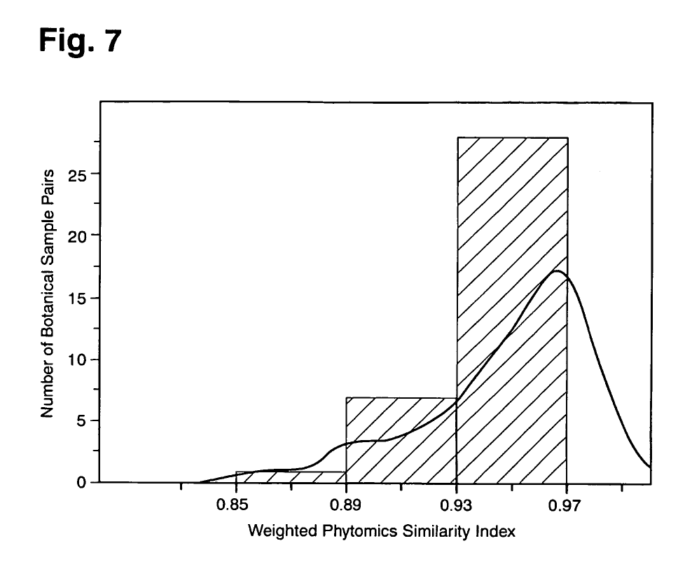

The matrix fingerprints and the associated analytical methods may be used to correlate or to determine quantitative equivalence of a specific batch of an herbal composition (single herb or multiple herbs of a formula) to a standardized, master, or batch of a same or substantially similar herbal composition. In addition, it can be used to rapidly identify datapoints (chemical compounds or biological responses) that are poorly correlated and to probe the basis for the poor correlation. We use as an example the comparison of nine batches sourced from various places in China and Taiwan of Huang Qin (Scutellariae Radix) and analyzed by LC/MS. Utilizing a consensus set of 46 LC/MS peaks, pair wise average PSI values can be computed. These values are found to range between 0.86 and 0.99 as seen in the pairwise comparison in Table 4 and plotted in FIG. 7.

TABLE 4
A table of weighted PSI values comparing pairwise, nine different
batches of standard extracts of Scutellaria Radix. Forty-six common peaks were used in
the comparison and PSI values range from a low of 0.86 to a high of 0.99. Individual
histograms of this data can be interrogated to find outliers, define classifications, identify
sub-sets of datapoints, correlate intra-relationships between datapoints etc.

| | SCUTE-1 | SCUTE-2 | SCUTE-3 | SCUTE-4 | SCUTE-5 | SCUTE-6 | SCUTE-7 | SCUTE-8 | SCUTE-9 |
|---|---|---|---|---|---|---|---|---|---|
| SCUTE-1 | | 0.86 | 0.89 | 0.93 | 0.92 | 0.89 | 0.93 | 0.91 | 0.89 |
| SCUTE-2 | | | 0.97 | 0.95 | 0.95 | 0.92 | 0.94 | 0.96 | 0.98 |
| SCUTE-3 | | | | 0.96 | 0.96 | 0.94 | 0.97 | 0.97 | 0.99 |
| SCUTE-4 | | | | | 0.98 | 0.94 | 0.97 | 0.96 | 0.96 |
| SCUTE-5 | | | | | | 0.97 | 0.98 | 0.97 | 0.97 |
| SCUTE-6 | | | | | | | 0.97 | 0.95 | 0.94 |
| SCUTE-7 | | | | | | | | 0.97 | 0.97 |
| SCUTE-8 | | | | | | | | | 0.97 |
| SCUTE-9 | | | | | | | | | |

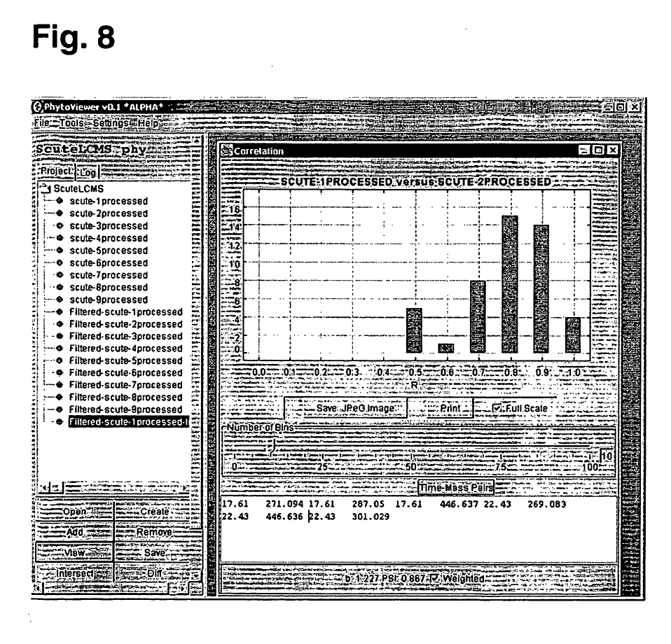

It should be noted that multiple injections of the same batch of the botanical, produces a PSI score of 0.99—nearly identical matches. From these plots, one can then begin to analyze the cut-off criteria that should be used to define specifications that can separate an acceptable lot from a non-acceptable lot. With a limited number of samples we would select a PSI score of 0.9 for this particular botanical. With the weighting function employed, one can define which datapoints contribute most to the PSI comparison, based on datapoint importance, datapoint value confidence etc. Examination of any one of these pairs of botanicals in more detail reveals a histogram of PSI values for individual datapoints (LC/MS peaks). This histogram can then be queried to identify which of the LC/MS peaks is responsible for the low correlation, as shown in FIG. 8.

### Example 6

### Quality Control (Raw Botanical and Process Treatment)

Raw botanicals can vary tremendously based on growing season, geographic location, plant age, plant part, rain fall, fertilizer, amount of light, etc. In addition, botanicals can be processed from their raw state by a variety of defined traditional and modern methods including pretreatments (soaking, roasting, drying, frying, honey treatment, etc.), storage conditions (time, temperature, etc.), extraction solvents (water (cold, hot), alcohol, acid, liquid gases, organic solvents, etc.), extraction conditions (time, mixtures, temperature, etc.), post-extraction treatments (spray dry, rotary evaporation, acid treatment, excipient addition, etc.), etc. Each of these methods in the manufacturing process can and does alter the chemical composition and possibly the biological activity. The matrix method provides a comprehensive approach to monitoring such changes. An example of a proprietary post-treatment is given as an illustration (Table 5) using the nine samples of Scutellaria Radix before and after treatment.

TABLE 5
A list of weighted PSI values comparing untreated and
post-treated extracts of Scutellaria Radix. The post-treatment
mimics the normal digestive processes that can alter the
chemical nature and balance within a multi-mixture botanical
extract. The data indicate that some batches are more
susceptible than other batches and can lead to the identification
of the sets of molecules responsible for the susceptibility.

| Sample | PSI-Value |
|---|---|
| scute1 | 0.78 |
| scute2 | 0.95 |
| scute3 | 0.93 |
| scute4 | 0.86 |
| scute5 | 0.94 |
| scute6 | 0.92 |
| scute7 | 0.60 |
| scute8 | 0.68 |
| scute9 | 0.75 |

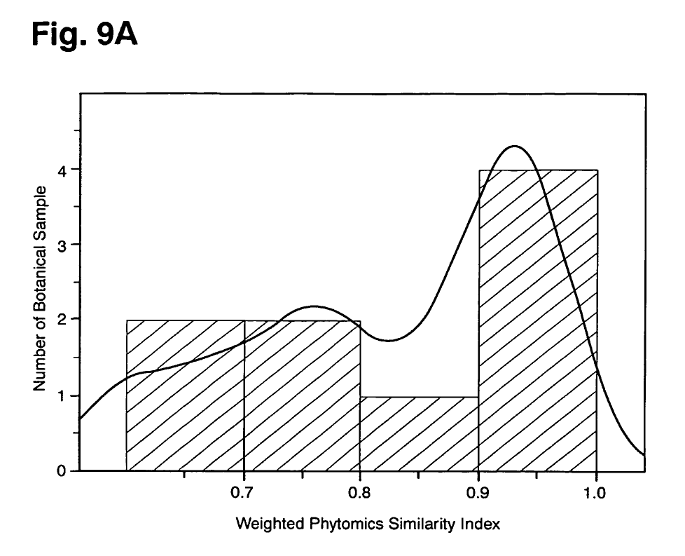

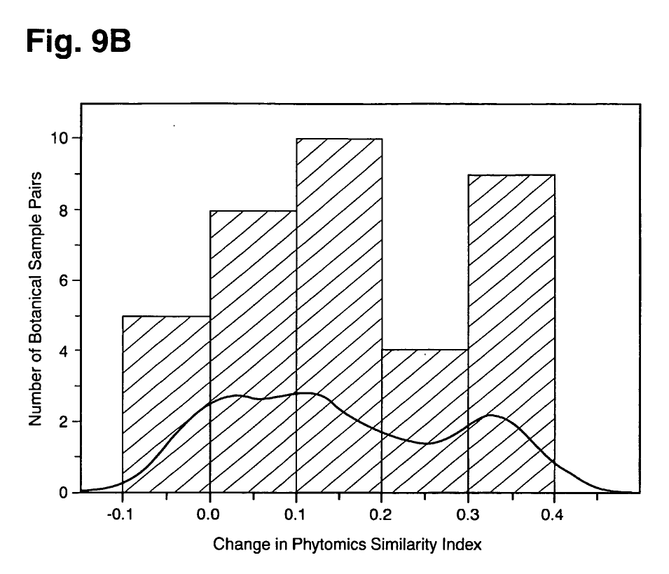

This treatment was designed to mimic components of normal digestive processes for orally taken products. In our case, this proprietary treatment modifies significantly the chemical composition and significantly reduces the similarity. When analyzed by the PSI method we can identify the subset of molecules using the proprietary PhytoViewer software that are invariant and the overall susceptibility of the sample to the treatment. Differences in PSI values range between 0.1 and 0.4 and when plotted as a histogram (see FIG. 9 and its accompanying description), indicate a cut-off in the PSI-difference of 0.2 between susceptible and non-susceptible batches.

### Example 7

### Quality Control (Biological Response)

A critical evaluation of any biological assay is the reproducibility of the assay itself. The PSI analysis can be useful in evaluating the effects of a single batch of a botanical (or single molecule) on the biological response. For example, consider a list of up and down regulated genes (Affymetrix™ U133A chips processed at core facilities at Yale University and Stony Brook) from six independent treatments of a Jurkat cell line of a single batch of an herbal formulation PHY906. A consensus set of 70 genes (55 up regulated and 15 down regulated) were culled from the data and used to compute the matrix and determine PSI values (Table 6).

TABLE 6
A table of weighted PSI values comparing pairwise, six different
genomic array experiments of Jurkat cells treated with identical
PHY906 extracts or left untreated to generate the signal log ratio
value used in the matrix. The PSI values indicate the level of
accuracy of different cell culture, gene array facility and chip
variability in the overall gene expression pattern. A total of
70 common genes between the six repeat data sets were
used in this comparison.

| | Repeat- | Repeat- | Repeat- | Repeat- | Repeat- | Repeat- |
|---|---|---|---|---|---|---|
| | 1 | 2 | 3 | 4 | 5 | 6 |
| Repeat-1 | | 0.91 | 0.942 | 0.951 | 0.912 | 0.913 |
| Repeat-2 | | | 0.883 | 0.912 | 0.907 | 0.903 |
| Repeat-3 | | | | 0.913 | 0.925 | 0.856 |
| Repeat-4 | | | | | 0.881 | 0.915 |
| Repeat-5 | | | | | | 0.845 |
| Repeat-6 | | | | | | |

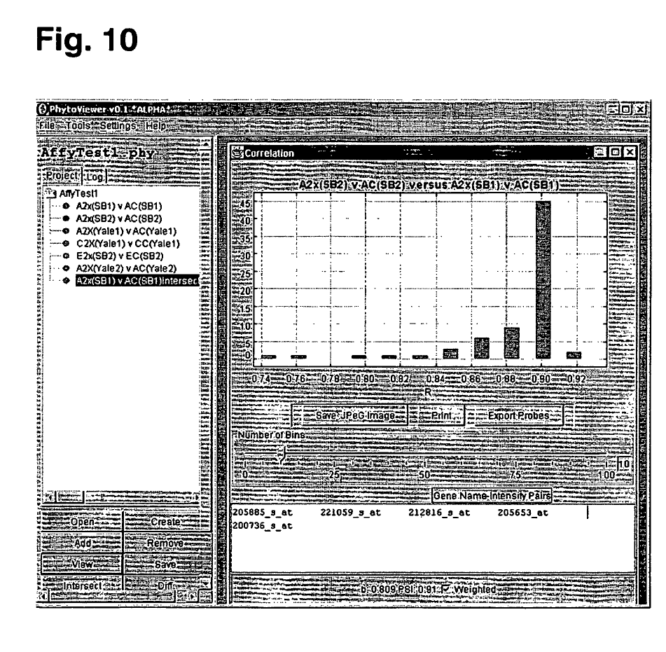

With the only variables being cell culture variation, chip reproducibility and test facility accuracy, these results can be useful to define that PSI values of 0.85 or higher are within experimental error that can establish a benchmark for determining a biological equivalency for consistency. In addition, outliers from the histogram of PSI values for individual genes (see FIG. 10 and the accompanying description) indicates a small set of genes with considerable divergence in the intra-ratio balance with other genes.

This may help to define which of the consistently observed genes are most stable in comparison with the gene response profile of all other genes and hence should be included or excluded from the signature gene bioresponse set for that particular botanical. Similar to the chemical fingerprint example (FIG. 5) and its use in defining the similarity of the chemical composition between botanicals, the bioresponse matrix fingerprint can also be used as a quality control readout of the effects of the chemical components on the genomic levels. For example, an ensemble of cells, each characterized by their activity with a botanical, can be arranged in a vector form. Therefore, each botanical will have a unique vector of biological significance associated with it. Genomic data also provides a powerful signature of the biological response of a botanical substance. DNA microarrays allow one to correlate gene expression profiles of cell activity with a particular botanical drug activity. The degree of correlation on the basis of botanicals and the basis of genes can be assessed. The result of this analysis is, for every botanical, a vector of correlation scores with every gene in the dataset. Representing each botanical with a vector of gene expression correlations provides a highly specific bioresponse fingerprint of the botanical. As an example, a Jaccard similarity index would determine the similarity of two botanicals based on their biological responses. In this way, a large dataset of botanicals could quickly be pruned into a biorelevant subset, for further comparison with other fingerprinting methods, e.g. LC/MS.

Proteomics addresses the actual expressed levels of protein in the cell and is a valuable complement to genomics profiling. A SELDI-MS experiment that detects the amount of protein bound to a particular surface substrate is used to illustrate that profound changes in the protein bioresponse profile can be quantitated using the matrix method and the PSI values. Jurkat cells were treated with three different doses of the botanical extract, PHY906, and the protein response monitored 24 hours later. The matrix of PSI values (Table 7) indicates that even low doses of the PHY906 can cause significant changes (0.83-0.85) but that major changes occur between doses of 0.1 and 1.0 mg/ml of PHY906 (0.38-0.49).

TABLE 7
A table of weighted PSI values comparing pairwise
four proteomic patterns (Ciphergen data using SELDI
method and the IMAC chip) of different doses (0.0, 0.02, 0.1
and 1.0 mg/ml) of PHY906 on Jurkat cells. The PSI values
indicate a quantitative difference of the pattern and ratio
pattern of expressed proteins between various treatments and
indicate that the largest dose response change in the protein
expression levels occur between 0.1 mg/ml and 1.0 mg/ml.

| | | | Dose | Dose |
|---|---|---|---|---|
| | Control | Dose 0.02 mg/ml | 0.1 mg/ml | 1.0 mg/ml |
| Control | 1 | 0.85 | 0.83 | 0.49 |
| Dose 0.02 mg/ml | | 1 | 0.71 | 0.38 |
| Dose 0.1 mg/ml | | | 1 | 0.4 |
| Dose 1.0 mg/ml | | | | 1 |

Because protein levels tend to be correlated in a living cell to provide a level of dynamic homeostasis, this method including the off-diagonal ratio terms, allows for the inclusion of protein change correlations and of determining the clusters of protein changes more rapidly.

### Example 8

### Improving an Herbal Composition or Identifying New Therapeutic Uses for an Herbal Composition

The matrix method can also be used to correlate the biological response fingerprint matrix with the chemical component fingerprint matrix to identify patterns of molecular species that may be responsible for a complex pattern of bioresponses. This concept of a systems biology approach to analyze complex multicomponent mixtures, requires pattern recognition and intra-dependent data analysis as embodied in the matrix method. With the approach of combining chemical and biological response fingerprints, one will be able to define biologically silent or inactive molecules and patterns of biologically relevant chemical components that will help to refine the bioactive nature of the mixture. This information may lead to improved botanical compositions or novel formulations by the creation of botanical analogs (substitutions, deletions and ratio adjustments of existing formulations). Similarly, treatment of cell culture or animals with botanicals of unknown or multiple claims (which is usually the case) and subsequent analysis of the bioresponse pattern, may indeed lead to insights into new claims. An example of a botanical drug, PHY906, claimed for diarrhea, also indicated in a broad screen chemokine response panel the down regulation of the cytokine IL-5, which is strongly implicated in the inflammatory processes of asthma. This finding, a consequence of examining the matrix fingerprint, further correlated the effects with IL-6 and other cytokines and opened up new utility avenues for the PHY906 drug.

### Example 9

### Characterizing An Unknown Herbal Medicine

Often traditional Chinese medicines (TCMs) are composed of multiple botanicals and maintained as family or trade secrets. Analysis of the samples can reveal the chemical components and be used to identify the botanical ingredients, the ratios of ingredients and even the manufacturing process by using the matrix fingerprint. Simply evaluating the chemical components individually may be sufficient to identify the individual ingredients. However, the ratios of ingredients and more subtly the source of raw botanicals and the manufacturing process can greatly alter the chemical component balance in a more complex, non-linear manner. This ratio balance, and the component intra-relationship pattern can be used as a superior means to fully characterize the nature of the product. As noted, analysis of the chemical fingerprint by this method could establish chemical equivalency between samples. Simulations of pattern matching could be used to define the ratio of botanical used in the final product. Once established, the ratio of the botanicals in the final composition could be subjected to a selection of extraction methods in a systematic way to deduce and to steer the optimization manufacturing process that brings the two patterns of phytochemicals into agreement. This can only be done effectively by focusing on the overall pattern of phytochemicals as opposed to following a small set of individual compounds. In addition to a chemical component matrix analysis, the biological response pattern could also be used to determine a more biorelevant comparison. In this case a bioequivalency would be established, by matching the enzyme/receptor, chemokine, proteomic, genomic, animal response and/or behavioral response through a systematic sampling of botanical extracts, botanical ingredients and manufacturing regimens.

The foregoing detailed description has been given for clearness of understanding only and no unnecessary limitations should be understood therefrom as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein before set forth and as follows in the scope of the appended claims.

1. A method of generating a matrix fingerprint representing the chemical and/or biological response properties of a herbal composition comprising obtaining appropriate data points for the herbal composition; digitizing the data points; and generating a matrix fingerprint for the herbal composition, wherein the matrix fingerprint comprises the digitized data.

2. The method of claim 1, wherein the matrix fingerprint is generated by placing the digitized data points along the matrix diagonal and placing the ratio of each digitized data point to every other digitized data point in the off-diagonal positions of the matrix.

3. A method of comparing the similarity between two or more herbal compositions comprising:
a) obtaining data points for the two or more herbal compositions;
b) digitizing the data points;
c) comparing the digitized data to determine those data points that the two or more herbal compositions have in common;
d) generating a matrix fingerprint for each herbal composition, wherein the matrix comprises the digitized data of the herbal composition for each of the common data points; and
e) comparing the similarity between the two or more herbal compositions by comparing the matrix fingerprints by a variety of statistical or rule-based methods.

4. The method of claim 3, wherein the matrix fingerprint for each of the two or more herbal compositions is generated by:
i) placing the ratio of each common digitized data point to every other common digitized data point in the off-diagonal positions of the matrix.

5. The method of claim 3 or 4, wherein the matrix fingerprints of the two or more herbal compositions are compared using set operations, statistical analysis or computational models.

6. The method of claim 5, wherein the statistical analysis is linear correlation.

7. A method of determining statistical classification models and for determining quality control criteria for two or more biological samples, said method comprising generating matrix fingerprints for the two or more biological samples; conducting statistical evaluations and comparisons for the two or more matrix fingerprints by computer-based algorithms to compute PSI values for individual data points; capturing a range of individual PSI values in a histogram or other visual display; using the display to identify poorly correlated data points; conduct a numerical analysis of the histogram and PSI values to determine statistical classification models and for determining quality control criteria.

8. The method of claim 7 wherein the computer-algorithms can be written in C++, Pearl, Java or other modern languages.

9. The method of claim 7 wherein the computer-algorithms can be conducted on a personal computer, a hand-held computer, a vector support machine, or a mainframe computer.

10. The method of claim 7 wherein the method is used to assist in quality control, classification, new drug identification, manufacturing, sample treatment processes, sample adulteration, sample tampering, and structure-biological activity correlation relationships of biological, herbal or multi-component samples.

11. The method of claim 7 wherein the method is used for the purpose of quality control, classification definition, new drug identification, new biological target identification, manufacturing differences, sample adulteration and tampering detection, structure-biological activity correlation relationships of the bioresponse of a single or multiple chemical component(s).
